(19) United States
(12) Patent Application Publication
(10) Pub. No.: US 2025/0249038 A1
(43) Pub. Date: Aug. 7, 2025

(54) CD30 HINGE AND/OR TRANSMEMBRANE DOMAIN-BASED CHIMERIC ANTIGEN RECEPTORS

(71) Applicant: BOARD OF REGENTS, THE UNIVERSITY OF TEXAS SYSTEM, Austin, TX (US)

(72) Inventors: Sattva S. NEELAPU, Bellaire, TX (US); Jingwei LIU, Houston, TX (US); Yongfu TANG, Houston, TX (US); Sridevi PATCHVA, Houston, TX (US)

(73) Assignee: BOARD OF REGENTS, THE UNIVERSITY OF TEXAS SYSTEM, Austin, TX (US)

(21) Appl. No.: 18/856,849

(22) PCT Filed: Apr. 26, 2023
(86) PCT No.: PCT/US2023/066246

§ 371 Date: Oct. 14, 2024

Related U.S. Application Data
(60) Provisional application No. 63/335,560, filed on Apr. 27, 2022.

(51) Int. Cl. A61K 35/17; A61K 40/11; A61K 40/31; A61K 40/42; A61K 45/06; A61P 35/00; C07K 14/705; C07K 14/725; C07K 16/28; C12N 5/0783

(52) U.S. Cl. CPC A61K 35/17; A61K 40/11; A61K 40/31; A61K 40/4211; A61K 40/4232; A61K 45/06; A61P 35/00; C07K 14/7051; C07K 14/70521; C07K 14/70578; C07K 16/2803; C07K 16/2875; C12N 5/0636; A61K 2239/13; A61K 2239/17; A61K 2239/21; A61K 2239/22; A61K 2239/48

(57) ABSTRACT

Aspects of the present disclosure include improvements to chimeric antigen receptor (CAR) constructs, CAR comprising immune cells, as well as methods for making such constructs and/or cells and methods for their use in treatment of disease (e.g., cancer). Disclosed are immune cells comprising CARs comprising CD30-derived hinge and/or transmembrane and methods for use of such cells in treatment of malignancies (e.g., B-cell malignancies). Also disclosed are polynucleotides encoding a CAR comprising CD30-derived hinge and/or transmembrane domains, as well as cells comprising such polynucleotides and pharmaceutical compositions comprising such cell

### CROSS-REFERENCE TO RELATED APPLICATIONS

This application claims priority to U.S. Provisional Patent Application Ser. No. 63/335,560 filed Apr. 27, 2022, which is incorporated by reference herein in its entirety.

### SEQUENCE LISTING

The instant application contains a Sequence Listing which has been submitted in ST26 format and is hereby incorporated by reference in its entirety. Said ST26 copy, created on Apr. 25, 2023, is named MDAC_1327WO_Sequence_Listing.xml and is 53,662 bytes in size.

### BACKGROUND

### I. Technical Field

This disclosure relates generally to at least the fields of cancer biology, immunology, and medicine.

### II. Background

Chimeric antigen receptor (CAR) cell therapy is generally regarded as an effective solution for relapsed or refractory tumors, particularly for hematological malignancies. Although commercial anti-CD19 CAR-T therapies have produced impressive outcomes, setbacks such as high relapse rates and resistance are common. There is a need to discover engineered CAR components that are more effective at improving effector cell therapeutic use, and to further improve patient outcomes.

### SUMMARY

Described herein are new functional components of a chimeric antigen receptor (CAR), CARs comprising one or more of the functional components, engineered cells comprising one or more of the functional components, compositions comprising one or more of the functional components, and methods of using the aforementioned.

Embodiments of the present disclosure include nucleic acids, polynucleotides, polypeptides, proteins, peptides, constructs, vectors, cells, therapeutic cells, immune cells, engineered cells, methods for generating engineered cells, methods for detecting engineered cells, methods for isolating engineered cells, methods for depleting engineered cells, and methods for purifying engineered cells. Nucleic acids of the disclosure may encode one or more polypeptides of the disclosure, including one or more functional components of a chimeric polypeptide. In some embodiments, a nucleic acid molecule of the disclosure encodes a chimeric polypeptide. In some embodiments, a nucleic acid molecule of the disclosure encodes two or more chimeric polypeptides. A chimeric polypeptide of the disclosure can include at least 1, 2, 3, or more of the following regions or domains: a signal peptide, an extracellular domain, a hinge region, a transmembrane domain, and an intracellular region. An engineered cell of the disclosure can comprise 1, 2, 3, 4, or more polynucleotides and/or polypeptides of the disclosure. Methods of the present disclosure can include at least 1, 2, 3, 4, or more of the following steps: introducing a polynucleotide into a cell, introducing a vector into a cell, introducing a polypeptide into a cell, expressing a polypeptide in a cell, expanding a population of cells, contacting a cell with an antigen-binding protein, contacting a cell with an antibody drug conjugate, and detecting a cell with an imaging agent.

In certain embodiments, the present disclosure provides a functional component of a Chimeric Antigen Receptor (CAR), wherein the functional component comprises a CD30 (also known as TNFRSF8, D1S166E, and Ki-1). hinge domain and/or a CD30 transmembrane domain. In certain embodiments, the present disclosure provides a CAR comprising: i) an antigen binding domain; ii) a CD30 hinge domain; iii) a CD30 transmembrane domain; iv) at least one intracellular costimulatory domain; and v) an intracellular stimulatory domain. In certain embodiments, a CD30 hinge domain comprises less than 51 contiguous amino acids and at least 7 contiguous amino acids of the extracellular domain of CD30. In certain embodiments, a CD30 transmembrane domain comprises no more than 27 contiguous amino acids of CD30. In certain embodiments, a CD30 hinge domain comprises less than 51 contiguous amino acids and at least 7 contiguous amino acids of the extracellular domain of CD30, and a CD30 transmembrane domain comprises no more than 27 contiguous amino acids of CD30.

In certain embodiments, a CD30 hinge and/or transmembrane domain do not comprise a cysteine. In certain embodiments, a CD30 hinge is at least 80%, 85%, 90%, 95%, or 98% identical to SEQ ID NO: 3. In certain embodiments, a CD30 transmembrane domain is at least 80%, 85%, 90%, or 95% identical to SEQ ID NO: 4. In certain embodiments, a CD30 hinge domain and/or transmembrane domain lack 3 or more contiguous amino acids according to SEQ ID NO: 6. In certain embodiments, a CD30 hinge domain comprises SEQ ID NO: 3. In certain embodiments, a CD30 transmembrane domain comprises SEQ ID NO: 4. In certain embodiments, a CD30 domain and transmembrane domain are encoded by a nucleotide sequence at least 75%, 80%, 85%, 90%, 95%, or 98% identical to SEQ ID NO: 39.

In some certain embodiments, the present disclosure provides a CAR comprising a CD30 hinge and/or transmembrane domain, and at least one antigen binding domain targeted to CD4, CD5, CD7, CD10, CD19, CD20, CD22, CD30, CD79a, CD79b, SLAM-F7, CD123, CD70, CD72, CD33, CD38, CD80, CD86, CD138, CLL-1, FLT3, ROR-1, TACI, TRBC1, MUC1, PD-L1, CD117, FR, LeY, HER2, IL13Ra2, DLL3, DR5, FAP, LMP1, MAGE-A1, MAGE-A4, MG7, MUC16, PMEL, ROR2, VEGFR2, AFP, EphA2, PSCA, EPCAM, EGFR, PSMA, EGFRVIII, GPC3, CEA, GD2, NY-ESO-1, TCL1, mesothelin, and/or BAFF-R. In certain embodiments, the present disclosure provides a CAR comprising a CD30 hinge and/or transmembrane domain, and at least one antigen binding domain targeted to CD19, CD20, CD22, CD70, CD79B, CD79A, ROR1, BCMA, BAFF receptor, GD2, and/or claudin18.2. In certain embodiments, an antigen binding domain is targeted to CD19, CD79B, and/or CD70.

In certain embodiments, the present disclosure provides a CAR comprising a CD30 hinge and/or transmembrane domain, and at least one intracellular costimulatory domain comprising a CD8, 4-1BB (CD137), CD27, CD28, CD30, OX-40 (CD134), CD38, CD35, CD45, CD4, CD5, CD9, CD16, CD22, CD33, CD37, CD64, CD80, CD86, or CD154, intracellular costimulatory domain. In certain embodiments, at least one intracellular costimulatory domain comprises CD28.

In certain embodiments, the present disclosure provides a CAR comprising a CD30 hinge and/or transmembrane domain, and at least one intracellular stimulatory domain comprising a DAP12, DAP10, FCER1G (Fc epsilon receptor I gamma chain), CD3δ (CD3 delta), CD3ε (CD3 epsilon), CD3γ (CD3 gamma), CD3ζ (CD3 zeta), or CD79A, intracellular stimulatory domain. In certain embodiments, at least one intracellular stimulatory domain comprises CD3ζ (CD3 zeta) intracellular stimulatory domain.

In certain embodiments, the present disclosure provides a CAR comprising a CD30 hinge and/or transmembrane domain, at least one intracellular costimulatory domain, and at least one intracellular stimulatory domain, wherein the polypeptide sequence comprising the same are at least 85%, 90%, 95%, 99%, or 100% identical to SEQ ID NO: 2.

In certain embodiments, the present disclosure provides a cell comprising CAR comprising a CD30 hinge and/or transmembrane domain. In certain embodiments, a cell is an immune cell. In certain embodiments, a cell is a T cell or an NK cell. In certain embodiments, a cell is derived from a healthy donor. In certain embodiments, a cell is derived from a patient. In certain embodiments, the T cells comprise CD4+ T cells, CD8+ T cells, iNKT cells, NKT cells, γδ T cells, regulatory T cells, innate lymphoid cells, or a combination thereof. In certain embodiments, the T cell is a γδ T cell. In certain embodiments, an immune cell is an immune cell as described in international publication number WO2021034982A1, which is incorporated herein by reference in its entirety for the purpose.

In some embodiments, the present disclosure provides a cell comprising a CAR and at least one additional transgene. In certain embodiments, the at least one additional transgene encodes an immunomodulatory gene. In some embodiments, an immunomodulatory gene is a survival-promoting gene. In some embodiments, an immunomodulatory gene is BCL6. In some embodiments, an immunomodulatory gene is an anti-apoptotic B-cell lymphoma 2 (BCL-2) family gene. In certain embodiments, the anti-apoptotic BCL-2 family gene is BCL2L1 (Bcl-xL), BCL-2, MCLI, BCL2L2 (Bcl-w), BCL2A1 (Bfl-1), BCL2L10 (BCL-B), or a combination thereof. In certain embodiments, the anti-apoptotic BCL-2 family gene is Bcl-xL.

In some embodiments, a cell comprises at least one manmade mutation in an endogenous gene, at least one heterologous nucleic acid that can modify expression of at least one endogenous gene, and/or a nucleic acid that can exert anti-apoptotic function. In certain embodiments, an endogenous gene is an immunomodulatory gene. In certain embodiments, an endogenous gene is an apoptotic protein (e.g., Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, etc.). In certain embodiments, an endogenous gene is a pro-apoptotic gene (e.g., BCL2L11 (BIM), BBC3 (PUMA), PMAIPI (NOXA), BIK, BMF, BAD, HRK, BID, BAX, BAK1, BOK, etc.) In certain embodiments, a nucleic acid that can exert anti-apoptotic function comprises a sequence encoding IGF1, HSPA4 (Hsp70), HSPB1 (Hsp27), CLAR (cFLIP), BNIP3, FADD, AKT, and NF-κB, RAFI, MAP2K1 (MEK1), RPS6KA1 (p90Rsk), JUN, C-Jun, BNIP2, BAG1, HSPA9, HSP90B1, miRNA21, miR-106b-25, miR-206, miR-221/222, miR-17-92, miR-133, miR-143, miR-145, miR-155, miR-330, and/or any combination thereof.

In certain embodiments, a cell comprises at least one safety switch. In some embodiments, a safety switch is truncated EGFR (e.g., an EGFR lacking domains 1 and 2). In certain embodiments, a cell is an immune cell (e.g., T cells, innate lymphoid cells, and/or NK cells) that expresses IL-2, IL-15, other growth or differentiation factors, or a combination thereof.

In certain embodiments, a cell maintains a proliferation rate for at least 3 months, 4 months, 5 months, 6 months, 7 months, 8 months, 9 months, 10 months, 11 months, 12 months, or any range therebetween. In certain embodiments, cells have enhanced antitumor cytotoxicity, in vivo proliferation, in vivo persistence, and/or improved function.

In certain embodiments, provided herein are compositions comprising at least 50 million, 100 million, 200 million, 500 million, 750 million, 1 billion, 2 billion, 3 billion, 4 billion, 5 billion, 6 billion, 7 billion, 8 billion, 9 billion, or 10 billion immune cells, including T cells, innate lymphoid cells, NK cells, or a mixture thereof.

In certain embodiments, provided herein are vectors. In certain embodiments, a vector is a lentiviral vector. In certain embodiments, introducing a vector to a cell comprises transducing the cells with the lentiviral vector in the presence of IL-2 and/or other growth factor(s). In certain embodiments, IL-2 is at a concentration of 10 IU/mL to 1000 IU/mL, such as 10-50 IU/mL, 50-75 IU/mL, 75-100 IU/mL, 100-250 IU/mL, 250-500 IU/mL, 500-750 IU/mL, or 750-1000 IU/mL. In certain embodiments, IL-2 is at a concentration of 100, 200, 300, 400, or 500 IU/mL.

In certain embodiments, provided herein are methods for the treatment of an immune-related disorder, infectious disease, and/or cancer comprising treatment with at least one cell disclosed herein. In some embodiments, the disease or disorder is an infectious disease, cancer, and/or immune-related disorder. In certain embodiments, the immune-related disorder is an autoimmune disorder, graft versus host disease, allograft rejection, or other inflammatory condition. In some embodiments, the immune cells are allogeneic. In some embodiments, the immune-related disorder is a cancer. In some embodiments, the cancer is a solid cancer or a hematologic malignancy. In some embodiments, the cancer is a hematological malignancy.

In some embodiments, methods of treatment comprise treatment with at least one cell disclosed herein, and further comprises treatment with at least one additional therapeutic agent. In some embodiments, the at least one additional therapeutic agent comprises chemotherapy, immunotherapy, surgery, radiotherapy, drug therapy, hormone therapy, biotherapy, or a combination thereof.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1, depicts an exemplary lentiviral vector map used to select different hinge, different transmembrane domain, and/or different costimulatory domain combinations. Different hinge, transmembrane, and/or costimulatory domain combinations were cloned between the FMC63 scFV and the CD35 (CD3 zeta) domain to build different CARs. The plasmids expressing different CARs were transfected into 293T cells using lipofectamine 3000 for screening of CARs with acceptable CD19 antigen binding ability. LTR=long terminal repeat; HTM=hinge and transmembrane domain; costim=costimulatory domain.

FIG. 2, depicts exemplary Lentiviral vector maps of CD19-targeting CARs used to transduce T cells (e.g., infinite γδ T cells) generated from healthy donor T cells. sEFla=short (weak) EF1alpha promoter; tPGK=TetO-PGK promoter; HTM=hinge and transmembrane domain; costim=costimulatory domain; tCD34=cytoplasmic tail truncated CD34.

FIGS. 3A-3B, depicts the expression of anti-CD19 CARs with different hinge and transmembrane domains in T cells (e.g., infinite γδ T cells). 3A) T cells (e.g., infinite γδ T cells) cells generated from healthy donor T cells were transduced with lentiviral vectors expressing anti-CD19 CARs with CD28 hTM-CD28 costim, PDL1hTM-CD28 costim, and CD30hTM-CD28 costim. The CAR expression cassette was driven by a weak (short EF1a promoter) or a strong promoter (the PDL1 hTM CAR used the MSCV promoter, the CD30 hTM CAR used a composite PGK promoter). Anti-CD19 CAR expression was determined by staining with FITC-conjugated CD19 antigen. All the cells were sorted using the FITC conjugated CD19 antigen. The flowcytometry data was analyzed using FlowJo software. All constructs showed clear CAR positive populations before and after sorting, but the CD30 hTMCD28 costim CAR with TRE-PGK promoter showed the highest expression. 3B) Median fluorescence intensity (MFI) of CAR positive populations from transduced cells (quantification of 3A). For the two weak promoter-driven CARs, the MFI of CD30 hTM-CD28 costim CAR population was higher than the CD28 hTM-CD28 costim CAR before and after sorting. For the two strong promoter-driven CARs, the MFI of CD30 hTM-CD28 costim CAR population was higher than the PDL1 hTM-CD28 costim CAR before and after sorting. Data was representative of one of three independent experiments. HTM=hinge and transmembrane domain; costim=costimulatory domain.

FIG. 4, depicts cytotoxic activity of T cells comprising anti-CD19 CAR with CD30hTM-CD28-CD3z against cancer cells. T cells (e.g., infinite γδ T cells) generated from healthy donor T cells were transduced with lentiviral vectors expressing CD30hTM-CD28 costim-based anti-CD19 CAR (tPGK promoter). The CAR expression cassette was driven by a strong composite PGK promoter. After expansion of transduced T cells, the CAR positive percentage was about 20% prior to sorting. These CAR T cells (e.g., CAR infinite γδ T cells) were cocultured with Nalm6 tumor cells with 200 IU of exogenous IL-2 in the medium and percentage change in Nalm6 cells was monitored over 3 days. The results showed that the percentage of live Nalm6 tumor cells decreased rapidly over 3 days. HTM=hinge and transmembrane domain; costim=costimulatory domain. tPGK=TetO-PGK promoter.

FIGS. 5A-5C, depict comparisons of cytotoxic activity of anti-CD19 CARs with different hinge and transmembrane domains. T cells (e.g., infinite γδ T cells) generated from healthy donor T cells were transduced with lentiviral vectors expressing anti-CD19 CARs with CD8hTM-CD28 costim, CD28 hTM-CD28 costim, PDL1hTM-CD28 costim, CD30hTM-CD28 costim (see FIG. 2 for vector maps). After sorting, the CAR positive cells were cocultured with RFP-Luciferase expressing Nalm6 tumor cells in duplicate wells. The absolute number of live Nalm6 cells was calculated using CountBright™ Absolute Counting Beads on day 0, day 1 and day 2. 5A) shows that the CD28 hTM-CD28 costim based anti-CD19 CAR inhibited the proliferation of Nalm6 cells, better than the CD8hTM-CD28 costim based anti-CD19 CAR when cocultured without exogenous IL-2 in the medium. For coculture experiments with 200 IU/mL IL-2 in the medium, 5B) shows that CD30hTM-CD28 costim based anti-CD19 CAR had stronger cytotoxicity than CD28hTM-CD28 costim CAR. 5C) showed that CD30hTM-CD28 costim based anti-CD19 CAR had stronger cytotoxicity than PDL1hTM-CD28 costim CAR. Data was representative of one of two independent experiments. Statistical significance (P<0.05) between experimental conditions was determined by paired t-test. HTM=hinge and transmembrane domain; costim=costimulatory domain.

FIG. 6, depicts expression of anti-CD79B CARs with CD30 hinge and transmembrane domains. 293T cells were transfected with lentiviral plasmid expressing CD30 hTM-CD28 costim anti-CD79B CARs-one contains an scFv from SN8 clone of CD79B antibody, the other contains an scFv from the 2F2 clone of CD79B antibody. The CAR expression cassettes were all driven by composite human PGK promoter. Twenty-four hours after transfection, anti-79B CAR expression was determined by staining with APC conjugated CD79B antigen. The flow cytometry data was analyzed using FlowJo software. The results showed that the CD30 hinge and transmembrane domains also worked well with scFvs derived from other antibodies (e.g., not just those targeting CD19 using FMC63 scFv antibody). HTM=hinge and transmembrane domain; costim=costimulatory domain.

FIG. 7, depicts expression of anti-CD19 CARs with different hinge and transmembrane domains in 293T cells. 293T cells were transfected with lentiviral plasmid expressing anti-CD19 CARs comprising different hinge domain, transmembrane domain, and/or costimulatory domains. The CAR expression cassettes were all driven by MSCV promoter (see FIG. 1 for vector map). Transfection efficiency was determined by AF647 conjugated anti-EGFR antibody, and anti-CD19 CAR expression was determined by staining the transduced 293T cells with FITC conjugated CD19 antigen. The data showed that the hinge and transmembrane domain from some transmembrane receptors did not support optimal CAR expression (e.g., CD79A hTM-CD28 costim, Long CTLA4 hTM-CD28 costim, TIM3 hTM-CD28 costim). In addition, the data suggested that the entire construct with hinge and transmembrane domains and costimulatory domain together were critical for optimal CAR expression. This was based on the observation that combining CD28 costimulatory domain with PD1 or PDL1 hTM resulted in good CAR expression on cell surfaces, but not with CTLA4-, TIM3-, and CD79A-derived hTM domains. HTM=hinge and transmembrane domain; costim=costimulatory domain.

FIG. 8, depicts the signaling capability of anti-CD19 CARs with different hinge and transmembrane domains. CAR plasmids which showed acceptable expression in 293T cells were used to produce lentiviral vectors and transduced into Jurkat-Lucia™ NFAT reporter cell line (InvivoGen), which was used to quantify CAR-induced signaling by measuring luciferase activity. After sorting the CAR positive populations, each population was cocultured with Raji lymphoma cells at an Effector:Target (E:T) ratio of 1:1. After 24 hours, luciferase activity was measured in the supernatant as per manufacturer's instructions. The results showed that while all CARs could be specifically activated by Raji cells, CD30hTM-CD28 CAR induced the highest activity. Additionally, PD1hTM-CD28 CAR had higher tonic signaling than other CARs based on observed activity in the absence of Raji cells. Data was representative of one of two independent experiments. hTM=hinge and transmembrane domain; costi=costimulatory domain.

FIGS. 9A-9B, depict expression of anti-CD19 CARs with different hinge and transmembrane domains in infinite «β T cells. 9A) aß T cells generated from healthy donor T cells were transduced with lentiviral vectors expressing anti-CD19 CARs containing different hinge and transmembrane domain and costimulatory domains. All of the CAR expression cassettes were driven by MSCV promoter. Anti-CD19 CAR expression was determined by staining the transduced T cells with FITC conjugated CD19 antigen. CD30 hTM-OX40 costim CAR showed excellent CAR positive populations, similar to CD28 HTM-CD28 costim and CD8 HTM-BAFF-R costim CARs. CAR expression was low or absent with other constructs. 9B) Anti-CD19 CAR expression on transduced cells in 9A) was determined by staining with FITC conjugated CD19 antigen. The MFI of CD30 HTM-OX40 costim CAR+ population was the highest among all the constructs tested in this experiment. Together, these results suggested that the entire construct with hinge and transmembrane domains and costimulatory domain together were important for optimal CAR folding and surface expression. HTM=hinge and transmembrane domain; costim=costimulatory domain.

FIGS. 10A-10E, depict signaling capabilities of different CARs with CD30 hinge and transmembrane domain (HTM) and CD28-CD3z signaling domains. Lentiviral vectors expressing different CAR constructs were transduced into the Jurkat-Lucia™ NFAT reporter cell line, CAR+ cells were sorted, and CAR-induced signaling was quantified by measuring luciferase activity with or without coculture with Daudi lymphoma cells for 24 hours at an Effector:Target ratio of 1:1. 10A, 10B, and 10D) Showed that both FMC63 scFv-CD30HTM-CD28costim (CD19-CD30HTM-CD28 CAR) and SN8 scFv-CD30HTM-CD28costim (CD79b-CD30HTM-CD28 CAR) signaled only in the presence of cells that express CD19 and/or CD79b (e.g., Daudi tumor cells). 10C) Jurkat-Lucia™ NFAT reporter cells were also transduced with an Fc receptor CAR (FcR CAR-CD16V-CD30HTM-CD28). The data showed that the FcR CAR only signals in the presence of both rituximab (an anti-CD20 antibody) and Daudi tumor cells but not when co-cultured with Daudi tumor cells alone. 10E, scFv-CD30HTM-41BB CAR (CD19-CD30HTM-41BB CAR) was lentivirally transduced into the Jurkat-Lucia™ NFAT reporter cell line, CAR+ cells were sorted, and CAR-induced signaling was quantified by measuring luciferase activity with or without coculture with PDX203 lymphoma cells, a high-grade B-cell lymphoma cell line, developed in the inventors laboratory from a patient-derived xenograft. After 24 hours, luciferase activity was measured in the supernatant. The results showed that the CD19-CD30HTM-41BB CAR signaled only in the presence of PDX-203 tumor cells. Data was representative of one of two or three independent experiments. Overall, these results indicated that the CD30 HTM domain functioned as an efficient HTM component with multiple CAR designs targeting different antigens on tumor cells and/or with different costimulatory domains.

FIGS. 11A-11C, depict comparisons of cytotoxic activity of anti-CD19 CARs with CD28 or CD30 hinge and transmembrane (HTM) domains. T cells (e.g., infinite γδ T cells) generated from healthy donor M4 and donor M5 were transduced with lentiviral vectors expressing anti-CD19 CARs with a CD28 HTM-CD28 costimulatory domain or a CD30HTM-CD28 costimulatory domain respectively, cells were then sorted for CAR+ cells, and were cocultured with Luciferase-RFP-expressing Nalm6 acute lymphoblastic leukemia tumor cells in duplicate wells at an Effector:Target ratio of 5:1. The absolute number of live Nalm6 tumor cells was calculated using CountBright™ Absolute Counting Beads on day 0 and day 1, 11A) depicts changes in absolute numbers of live Nalm6 cells, and 11B) and 11C) depict the percentage change in live tumor cells. The results showed that the CD19 CAR with CD30 HTM domain had significantly stronger cytotoxicity than CD19 CAR with CD28 HTM domain. P values were calculated by unpaired t-test.

FIGS. 12A-12B, show the antitumor effects of scFv CD30HTM CAR-transduced infinite γδ CAR T cells in vivo. Luciferase-labeled Daudi Burkitt lymphoma tumor cells (2×10⁴ tumor cells/mouse) were injected intravenously into 3 groups of human IL-15 transgenic NSG mice (secreting physiological level of human IL-15) on day-2. Three infusions of infinite γδ T or infinite anti-CD19 CD30HTM-CD28Cos CAR-γδT were injected into the mice on Days 0, 3, and 8 at a dose of 8×10⁶ T cells/mouse/injection. The results showed that the CD30HTM-CD28Cos CAR-γδT slowed the progression of lymphoma. Tumor burden was assessed by bioluminescence imaging 12A) and mean total flux in each group was quantified 12B). The data indicated that the antitumor effect of infinite γδ CAR T cells was higher than infinite γδ T cells without a CD30HTM-CAR.

FIGS. 13A-13B, show the transduction, signaling, and CD70 binding abilities of truncated CD27 (tCD27) CAR with CD30 hinge and transmembrane domains. 13A), the CD27-based anti-CD70 CAR was made by fusion of the truncated CD27 extracellular domain (SEQ ID NO: 48) with the CD30 hinge and TM domains, CD28 costimulatory domain, and CD3z signaling domain. The signaling capability was determined using Jurkat-Lucia™ NFAT reporter cell line (Invivogen). Lentiviral vectors expressing this CAR construct was transduced into the Jurkat-Lucia™ NFAT reporter cell line, CAR+ cells were sorted, and CAR-induced signaling was quantified by measuring luciferase activity with or without coculture with a CD70 positive T cell line at an Effector:Target ratio of 1:1. After 24 hours, luciferase activity was measured in the supernatant. As shown, the tCD27-CD30HTM-CD28cos-CD3z CAR signaled only in the presence of cells that expressed CD70. The results indicated that the CD30 HTM domain functioned as an efficient HTM component with multiple CAR designs targeting different antigens on tumor cells and/or with different costimulatory domains. RLU=relative light units. 13B), shows expression of the aforementioned lentiviral vectors (tCD27-CD30HTM-CD28cos-CD3z CAR) transduced into primary human T cells. Ten days after transduction, CD27 and CD70 cell surface expression on CAR-T cells was measured. The CAR's ability to bind to CD70 was also tested for by staining with a fluorochrome conjugated recombinant CD70 protein. Non-transduced T cells and a low affinity anti-CD70 scFv antibody (Clone 1F6) were utilized as controls. The anti-CD27 antibody staining results indicated that tCD27 folded appropriately and was expressed on the surface of primary T cells (top row). The CD70 protein staining indicated that the tCD27 CAR bound recombinant CD70 protein (middle row). The anti-CD70 antibody staining showed that the CD70+ cells were nearly absent in tCD27 CAR-transduced T cells, indicating that cells expressing CD70 were either efficiently eliminated, or that CD70 on cell surfaces was masked through in cis binding (bottom row).

### DETAILED DESCRIPTION

Herein the inventors describe a number of studies that involved testing scores of hinges and/or transmembrane domains for suitability in CAR construction. Surprisingly, the inventors show that relatively few hinges and/or transmembrane domains can be used to build functional and/or effective CAR molecules. Among the examples of components tested, hinge and/or transmembrane domains derived from CD30 were efficacious. While not being limited by theory, one reason for this observed efficacy may be the underlying primary amino acid sequence of the CD30 hinge and/or transmembrane domains. In some embodiments, the CD30 derived hinge and/or transmembrane domains have more amino acids that provide flexibility (e.g., G or S) than those derived from CD28 or CD8a, the most commonly used hinge and/or transmembrane domains in CARs. Furthermore, in some embodiments, the described CD30-derived hinge and/or transmembrane domains do not comprise cysteines (C) in either the hinge or transmembrane region. In contrast, the CD28 hinge and transmembrane domains have 2 cysteines, and the CD8a hinge and transmembrane domains have 3 cysteines. It is known that cysteine-containing proteins or peptides tend to form homodimers between themselves or form heterodimers with other transmembrane proteins, which can result in unpredictable consequences for CAR expression and folding of the antigen binding domain (e.g. scFv) of the CAR molecule. Thus, the unique sequence of CD30-derived hinge and/or transmembrane domains potentially allows for better folding of the antigen binding domain (e.g. scFv) of the CAR molecule, accounting for the observed increases in cell surface expression of CD30 hinge and/or transmembrane domain comprising CARs as described herein. Consistent with this, results described herein showed that an anti-CD19 CAR comprising CD30 h/TM-CD28-CD3z has strong surface expression in T cells, robust signaling capability in a Jurkat reporter cell line (e.g., highest signaling capacity of the vectors tested), and exhibited potent cytotoxicity against B-cell leukemia.

The discovery of a new efficacious hinge and/or transmembrane domain provides the field with an additional tool with which, in certain embodiments, can be utilized to build dual, triple, quadruple, etc. CARs targeting two or more different antigens, as it is known that identical DNA sequences in a lentiviral vector can detrimentally affect the underlying constructs stability. Thus, the significantly different DNA sequence encoding the described CD30 hinge and/or transmembrane domain, compared to the established CD8a or CD28 hinge and transmembrane domains, facilitates production of stable lentiviral vectors to generate bi-, tri-, etc.-specific CARs to target more than one antigen simultaneously.

Aspects of the disclosure relate to improvements in CAR construct components (e.g., domains and/or regions). In certain embodiments, the disclosure provides a novel hinge and/or transmembrane (TM) domain which can be used to build chimeric antigen receptors (CAR). In certain embodiments, the hinge and/or TM domain is derived partially from the extracellular domain and complete transmembrane domain of human CD30 molecule. In certain embodiments, when connected with a costimulatory domain such as, but not limited to, OX40 or CD28, the CD30 hinge and/or TM domain can support strong CAR expression and effector cell cytotoxicity against tumor cells.

In some embodiments, CAR constructs comprising CD30 h/TM-CD28-CD3z can induce stronger CD3z downstream signaling in a Jurkat-Lucia™ reporter cell line (as one example) than comparable CAR constructs comprising CD28 h/TM-CD28-CD3z. In some embodiments, immune cells comprising CAR constructs comprising CD30 h/TM-CD28-CD3z have better cytotoxicity against cancer cells when compared to immune cells comprising CAR constructs comprising CD28 h/TM-CD28-CD3z.

### I. Polypeptides

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-52.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs: 1-38, 43, 45, and 47-50.

In some embodiments, the protein, polypeptide, or nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs: 1-38, 43, 45, and 47-50.

In some embodiments, the polypeptide, protein, or nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of SEQ ID NOs: 1-52 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 1-52.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS: 1-52 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS: 1-52.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in certain embodiments of compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Sequences

The amino acid sequence of certain polypeptides, including chimeric antigen receptors and portions, regions, and/or domains thereof, are provided in Table 1.

TABLE 1
amino acid sequences

| | SEQ ID | |
|---|---|---|
| Name | NO: | Sequence |
| CD30 hinge and | 1 | APASTSPTQSLLVDSQASKTLPIPTSAPVALSST |
| Transmembrane (TM) | | GKPVLDAGPVLFWVILVLVVVVGSSAFLL |
| domain | | |
| CD30 hinge and TM | 2 | APASTSPTQSLLVDSQASKTLPIPTSAPVALSST |
| domain with cytoplasmic | | GKPVLDAGPVLFWVILVLVVVVGSSAFLLYCWVR |
| spacer, CD28 costimulatory | | SKRSRLLHSDYMNMTPRRPGPTRKHYQPYAPPRD |
| domain, and CD3ζ | | FAAYRSRVKFSRSADAPAYQQGQNQLYNELNLGR |
| signaling domain | | REEYDVLDKRRGRDPEMGGKPRRKNPQEGLYNEL |
| | | QKDKMAEAYSEIGMKGERRRGKGHDGLYQGLSTA |
| | | TKDTYDALHMQALPPR |
| CD30 hinge domain | 3 | APASTSPTQSLLVDSQASKTLPIPTSAPVALSST |
| | | GKPVLDAG |
| CD30 TM domain | 4 | PVLFWVILVLVVVVGSSAFLL |
| CD30 hinge extracellular | 5 | APPLGTQPDCNPTPENGE |
| flanking region | | |
| CD30 full extracellular | 6 | FPQDRPFEDTCHGNPSHYYDKAVRRCCYRCPMGL |
| domain | | FPTQQCPQRPTDCRKQCEPDYYLDEADRCTACVT |
| | | CSRDDLVEKTPCAWNSSRVCECRPGMFCSTSAVN |
| | | SCARCFFHSVCPAGMIVKFPGTAQKNTVCEPASP |
| | | GVSPACASPENCKEPSSGTIPQAKPTPVSPATSS |
| | | ASTMPVRGGTRLAQEAASKLTRAPDSPSSVGRPS |
| | | SDPGLSPTQPCPEGSGDCRKQCEPDYYLDEAGRC |
| | | TACVSCSRDDLVEKTPCAWNSSRTCECRPGMICA |
| | | TSATNSCARCVPYPICAAETVTKPQDMAEKDTTF |
| | | EAPPLGTQPDCNPTPENGEAPASTSPTQSLLVDS |
| | | QASKTLPIPTSAPVALSSTGKPVLDAG |
| CD28 hinge and TM | 7 | IEVMYPPPYLDNEKSNGTIIHVKGKHLCPSPLFP |
| domain | | GPSKPFWVLVVVGGVLACYSLLVTVAFIIFWV |
| CD8 hinge and TM domain | 8 | TTTPAPRPPTPAPTIASQPLSLRPEACRPAAGGA |
| | | VHTRGLDFACDIYIWAPLAGTCGVLLLSLVIT |
| Fc Receptor CAR | 9 | MLLLVTSLLLCELPHPAFLLIGMRTEDLPKAVVF |
| | | LEPQWYRVLEKDSVTLKCQGAYSPEDNSTQWFHN |
| | | ESLISSQASSYFIDAATVDDSGEYRCQTNLSTLS |
| | | DPVQLEVHIGWLLLQAPRWVFKEEDPIHLRCHSW |
| | | KNTALHKVTYLQNGKGRKYFHHNSDFYIPKATLK |
| | | DSGSYFCRGLVGSKNVSSETVNITITVSSAPAST |
| | | SPTQSLLVDSQASKTLPIPTSAPVALSSTGKPVL |
| | | DAGPVLFWVILVLVVVVGSSAFLLYCWVRSKRSR |
| | | LLHSDYMNMTPRRPGPTRKHYQPYAPPRDFAAYR |
| | | SRVKFSRSADAITSYEKSDGVYTGLSTRNQETYE |
| | | TLKHEKPPQAIRKRQPEETNNDYETADGGYMTLN |
| | | PRAPTDDDKNIYLTLPPNDHVNSNNYTGLSTRNQ |
| | | ETYETLKHEKPPQ |
| ITAM containing domain | 10 | ITSYEKSDGVYTGLSTRNQETYETLKHEKPPQ |
| from Fc Receptor Gamma- | | |
| Chain | | |
| ITAM containing domain | 11 | YTGLSTRNQETYETLKHEKPPQ |
| from Fc Receptor Gamma- | | |
| Chain 2 | | |
| VSS-CD30 hinge and TM | 12 | VSSAPASTSPTQSLLVDSQASKTLPIPTSAPVAL |
| domain with | | SSTGKPVLDAGPVLFWVILVLVVVVGSSAFLLYC |
| immunoglobulin light chain | | WV |
| junction peptide linker | | |
| CD16 extracellular domain | 13 | GMRTEDLPKAVVFLEPQWYRVLEKDSVTLKCQGA |
| without CD16 hinge | | YSPEDNSTQWFHNESLISSQASSYFIDAATVDDS |
| | | GEYRCQTNLSTLSDPVQLEVHIGWLLLQAPRWVF |
| | | KEEDPIHLRCHSWKNTALHKVTYLQNGKGRKYFH |
| | | HNSDFYIPKATLKDSGSYFCRGLVGSKNVSSETV |
| | | NITIT |
| GM-CSF receptor signal | 14 | MLLLVTSLLLCELPHPAFLLI |
| peptide | | |
| CD28 costimulatory | 15 | RSKRSRLLHSDYMNMTPRRPGPTRKHYQPYAPPR |
| domain | | DFAAYRS |
| CD3z derived peptide | 16 | RVKFSRSADA |
| linker | | |
| ITAM containing domain | 17 | AIRKRQPEETNNDYETADGGYMTLNPRAPTDDDK |
| from CD32A intracellular | | NIYLTLPPNDHVNSNN |
| domain | | |
| CD19 CAR (FMC63 | 18 | MALPVTALLLPLALLLHAARPDIQMTQTTSSLSA |
| SCFV-CD30 HTM-CD28 | | SLGDRVTISCRASQDISKYLNWYQQKPDGTVKLL |
| cos-CD3Zeta) | | IYHTSRLHSGVPSRFSGSGSGTDYSLTISNLEQE |
| | | DIATYFCQQGNTLPYTFGGGTKLEITGGGGSGGG |
| | | GSGGGGSEVKLQESGPGLVAPSQSLSVTCTVSGV |
| | | SLPDYGVSWIRQPPRKGLEWLGVIWGSETTYYNS |
| | | ALKSRLTIIKDNSKSQVFLKMNSLQTDDTAIYYC |
| | | AKHYYYGGSYAMDYWGQGTSVTVSSAPASTSPTQ |
| | | SLLVDSQASKTLPIPTSAPVALSSTGKPVLDAGP |
| | | VLFWVILVLVVVVGSSAFLLYCWVRSKRSRLLHS |
| | | DYMNMTPRRPGPTRKHYQPYAPPRDFAAYRSRVK |
| | | FSRSADAPAYQQGQNQLYNELNLGRREEYDVLDK |
| | | RRGRDPEMGGKPRRKNPQEGLYNELQKDKMAEAY |
| | | SEIGMKGERRRGKGHDGLYQGLSTATKDTYDALH |
| | | MQALPPR |
| CD8a signal peptide | 19 | MALPVTALLLPLALLLHAARP |
| FMC63 scFv | 20 | DIQMTQTTSSLSASLGDRVTISCRASQDISKYLN |
| | | WYQQKPDGTVKLLIYHTSRLHSGVPSRFSGSGSG |
| | | TDYSLTISNLEQEDIATYFCQQGNTLPYTFGGGT |
| | | KLEITGGGGSGGGGSGGGGSEVKLQESGPGLVAP |
| | | SQSLSVTCTVSGVSLPDYGVSWIRQPPRKGLEWL |
| | | GVIWGSETTYYNSALKSRLTIIKDNSKSQVFLKM |
| | | NSLQTDDTAIYYCAKHYYYGGSYAMDYWGQGTSV |
| | | TVSS |
| CD30 hinge and TM | 21 | APASTSPTQSLLVDSQASKTLPIPTSAPVALSST |
| domain with | | GKPVLDAGPVLFWVILVLVVVVGSSAFLLYCWV |
| immunoglobulin light chain | | |
| junction peptide linker | | |
| CD3 signaling domain | 22 | RVKFSRSADAPAYQQGQNQLYNELNLGRREEYDV |
| | | LDKRRGRDPEMGGKPRRKNPQEGLYNELQKDKMA |
| | | EAYSEIGMKGERRRGKGHDGLYQGLSTATKDTYD |
| | | ALHMQALPPR |
| CD79B CAR (SN8 SCFV- | 23 | MALPVTALLLPLALLLHAARPDIVLTQSPASLAV |
| CD30 HTM-CD28 cos- | | SLGQRATISCKASQSVDYDGDSFLNWYQQKPGQP |
| CD3Zeta) | | PKLFIYAASNLESGIPARFSGSGSGTDFTLNIHP |
| | | VEEEDAATYYCQQSNEDPLTFGAGTELELKRSGG |
| | | GGSGGGGSGGGGSGGGGSEVQLQQSGAELMKPGA |
| | | SVKISCKATGYTFSSYWIEWVKQRPGHGLEWIGE |
| | | ILPGGGDTNYNEIFKGKATFTADTSSNTAYMQLS |
| | | SLTSEDSAVYYCTRRVPVYFDYWGQGTSVTVSSA |
| | | PASTSPTQSLLVDSQASKTLPIPTSAPVALSSTG |
| | | KPVLDAGPVLFWVILVLVVVVGSSAFLLYCWVRS |
| | | KRSRLLHSDYMNMTPRRPGPTRKHYQPYAPPRDF |
| | | AAYRSRVKFSRSADAPAYQQGQNQLYNELNLGRR |
| | | EEYDVLDKRRGRDPEMGGKPRRKNPQEGLYNELQ |
| | | KDKMAEAYSEIGMKGERRRGKGHDGLYQGLSTAT |
| | | KDTYDALHMQALPPR |
| SN8 scFv | 24 | DIVLTQSPASLAVSLGQRATISCKASQSVDYDGD |
| | | SFLNWYQQKPGQPPKLFIYAASNLESGIPARFSG |
| | | SGSGTDFTLNIHPVEEEDAATYYCQQSNEDPLTF |
| | | GAGTELELKRSGGGGSGGGGSGGGGSGGGGSEVQ |
| | | LQQSGAELMKPGASVKISCKATGYTFSSYWIEWV |
| | | KQRPGHGLEWIGEILPGGGDTNYNEIFKGKATFT |
| | | ADTSSNTAYMQLSSLTSEDSAVYYCTRRVPVYFD |
| | | YWGQGTSVTVSS |
| 4-1BB (CD137) costim | 25 | KRGRKKLLYIFKQPFMRPVQTTQEEDGCSCRFPE |
| | | EEEGGCEL |
| OX40 (CD134) costim | 26 | RRDQRLPPDAHKPPGGGSFRTPIQEEQADAHSTL |
| | | AKI |
| Immunoglobulin light | 27 | YCWV |
| chain junction region | | |
| derived peptide spacer | | |
| Full length extracellular | 47 | MARPHPWWLCVLGTLVGLSATPAPKSCPERHYWA |
| domain of CD27 including | | QGKLCCQMCEPGTFLVKDCDQHRKAAQCDPCIPG |
| an optional CD27 signal | | VSFSPDHHTRPHCESCRHCNSGLLVRNCTITANA |
| peptide (underlined) | | ECACRNGWQCRDKECTECDPLPNPSLTARSSQAL |
| | | SPHPQPTHLPYVSEMLEARTAGHMQTLADFRQLP |
| | | ARTLSTHWPPQRSLCSSDFIR |
| Truncated extracellular | 48 | MARPHPWWLCVLGTLVGLSATPAPKSCPERHYWA |
| domain of CD27 (tCD27) | | QGKLCCQMCEPGTFLVKDCDQHRKAAQCDPCIPG |
| including an optional CD27 | | VSFSPDHHTRPHCESCRHCNSGLLVRNCTITANA |
| signal peptide (underlined) | | ECACRNGWQCRDKECTECDPLP |
| CD70 CAR (tCD27-CD30 | 49 | MARPHPWWLCVLGTLVGLSATPAPKSCPERHYWA |
| HTM-CD28 cos-CD3Zeta) | | QGKLCCQMCEPGTFLVKDCDQHRKAAQCDPCIPG |
| | | VSFSPDHHTRPHCESCRHCNSGLLVRNCTITANA |
| | | ECACRNGWQCRDKECTECDPLPAPASTSPTQSLL |
| | | VDSQASKTLPIPTSAPVALSSTGKPVLDAGPVLF |
| | | WVILVLVVVVGSSAFLLYCWVRSKRSRLLHSDYM |
| | | NMTPRRPGPTRKHYQPYAPPRDFAAYRSRVKFSR |
| | | SADAPAYQQGQNQLYNELNLGRREEYDVLDKRRG |
| | | RDPEMGGKPRRKNPQEGLYNELQKDKMAEAYSEI |
| | | GMKGERRRGKGHDGLYQGLSTATKDTYDALHMQA |
| | | LPPR |
| CD27 signal peptide | 50 | MARPHPWWLCVLGTLVGLS |

### B. Variant Polypeptides

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within +2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### C. Chimeric Antigen Receptors

In some aspects, disclosed are chimeric antigen receptors (CARs). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a cancer antigen-specific CAR polypeptide, including in some cases a CAR that has been humanized to reduce immunogenicity (hCAR), comprising at least one intracellular signaling domain, a transmembrane domain, and an extracellular domain comprising one or more signaling motifs. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the CAR nucleic acids may be human genes used to enhance cellular immunotherapy for human patients. In a specific embodiment, the disclosure includes a full-length CAR cDNA or coding region. The antigen binding regions or domain can comprise a fragment of the VH and VL chains of a single-chain variable fragment (scFv) derived from a particular human monoclonal antibody (e.g., an anti-CD19 antibody such as FMC63.3 and/or an anti-CD79b antibody such as those described in PCT Patent Application Publication WO 2021/222944). In some embodiments, the fragment can also be any number of different antigen binding domains of a human antigen-specific antibody. In a more specific embodiment, the fragment is a cancer antigen-specific scFv encoded by a sequence that is optimized for human codon usage for expression in human cells. In some embodiments, an antigen binding region comprises a protein or polypeptide that acts as a ligand and/or receptor for another protein and/or polypeptide.

In some embodiments, an arrangement could be multimeric, such as a diabody or multimers. Multimers are most likely formed by cross pairing of the variable portion of the light and heavy chains into a diabody. The hinge portion of a construct can have multiple alternatives from being totally deleted, to having the first cysteine maintained, to a proline rather than a serine substitution, to being truncated up to the first cysteine. In some embodiments, an Fc portion can be deleted. In some embodiments, any protein that is stable and/or dimerizes can serve this purpose. In some embodiments, just one of the Fc domains, e.g., either the CH2 or CH3 domain from human immunoglobulin is utilized. In some embodiments, the hinge, CH2 and CH3 region of a human immunoglobulin that has been modified to improve dimerization can be utilized. In some embodiments, just the hinge portion of an immunoglobulin can be utilized.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding (e.g., anti-CD19, anti-CD79b, anti-CD70, etc.), or recognition component is linked to one or more transmembrane and intracellular signaling domains. In some embodiments, a CAR includes a transmembrane domain fused to an extracellular domain of the CAR. In some embodiments, a transmembrane domain that naturally is associated with one of the domains in the CAR is used. In some embodiments, a transmembrane domain is used that is not naturally associated with one of the domains of the CAR. In some instances, a transmembrane domain is selected or modified by amino acid substitution to avoid binding of such domains to the transmembrane domains of the same or different surface membrane proteins to minimize interactions with other members of the receptor complex. In some embodiments, a transmembrane domain is derived either from a natural or from a synthetic source. In some embodiments, where the source is natural, a transmembrane domain is derived from any membrane-bound or transmembrane protein. In some embodiments, transmembrane regions include those derived from (i.e. comprise at least the transmembrane region(s) of; also “from”) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD30, CD33, CD37, CD64, CD80, CD86, OX-40 (CD134), 4-1BB (CD137), CD154, ICOS/CD278, and so forth. In some embodiments, a transmembrane domain of the present disclosure is a transmembrane domain from CD8a. In some embodiments, a transmembrane domain of the present disclosure is a transmembrane domain from CD30.

In some embodiments, the CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB (CD137), OX-40 (CD134), ICOS, (CD278), CD30, HVEM, CD40, LFA-1 (CD11a/CD18), and ICAM-1, although in specific alternative embodiments any one of these listed may be excluded from use in a CAR.

In particular embodiments, specific CAR molecules are encompassed herein. In some cases, the antigen binding domain of the CAR is a scFv, and any scFv that binds to a cancer antigen may be utilized herein. In cases wherein an scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The scFv and/or ligand that binds the antigen in the CAR may or may not be codon optimized. In some embodiments, the antigen biding domain of a CAR is a ligand of another protein (e.g., a “bait” protein), such as a CD27 molecule acting to target CD70. In some embodiments, a CD27 molecule is a truncated CD27 extracellular binding domain (e.g., tCD27).

In particular embodiments, a vector encodes a cancer antigen-specific CAR and also encodes one or more other molecules. For example, a vector may encode both a first CAR (e.g., an anti-CD19 CAR) and a second CAR (e.g., an anti-CD79b CAR, anti-CD70 CAR, etc.).

In some embodiments, on the same molecule, the cancer antigen-specific CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens, there may be a linker between the two antigen-specific extracellular domains. Examples of CARs contemplated herein include, without limitation, CD19-specific (also “anti-CD19”) CARs, anti-CD70 CARs (also CD70 CARs or tCD27-CAR), and CD79b-specific (also “anti-CD79b”) CARs.

In particular embodiments of specific CAR molecules, a CAR may utilize CD28, DAP10, DAP12, 4-1BB, NKG2D, etc. or other costimulatory domains (which may be referred to herein as an intracytoplasmic domain). In some cases, CD3zeta is utilized without any costimulatory domains. In particular embodiments of specific CAR molecules, a CAR may utilize any suitable transmembrane domain, such as from CD30, DAP12, DAP10, 4-1BB, 2B4, OX40, CD27, NKG2D, CD8, CD28, IL12Rβ1, or IL12Rβ2. In particular embodiments of specific CAR molecules, a CAR may utilize a transmembrane domain from CD30.

### 1. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some embodiments, a signal peptide directs the nascent protein into the endoplasmic reticulum. This is essential if a receptor is to be glycosylated and anchored in the cell membrane. Generally, the signal peptide natively attached to the amino-terminal most component is used (e.g., in an scFv with orientation light chain-linker-heavy chain, the native signal of the light-chain is used).

In some embodiments, the signal peptide is cleaved after passage of the endoplasmic reticulum (ER), i.e., is a cleavable signal peptide. In some embodiments, a restriction site is at the carboxy end of the signal peptide to facilitate cleavage.

### 2. Antigen Binding Domain

Polypeptides of the present disclosure may comprise one or more antigen binding domains. An “antigen binding domain” describes a region of a polypeptide capable of binding to an antigen under appropriate conditions. In some embodiments, an antigen binding domain is a single-chain variable fragment (scFv) based on one or more antibodies (e.g., CD20 antibodies). In some embodiments, an antigen binding domain comprise a variable heavy (VH) region and a variable light (VL) region, with the VH and VL regions being on the same polypeptide. In some embodiments, the antigen binding domain comprises a linker between the VH and VL regions. A linker may enable the antigen binding domain to form a desired structure for antigen binding.

In certain embodiments of a chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD70, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, CD79a, CD79b, and so forth. In certain embodiments, a tumor associated antigen is CD19. In certain embodiments, a tumor associated antigen is CD79b. In certain embodiments, a tumor associated antigen is CD70. In certain embodiments, the CAR may be co-expressed with a cytokine to improve persistence when there is a low amount of tumor-associated antigen. For example, CAR may be co-expressed with IL-15.

In some embodiments, a CAR with a CD70 antigen binding domain comprises, consists of, or consists essentially of a sequence 75%, 80%, 85%, 90%, 95%, or 100% (and any value therebetween) identical to SEQ ID NO: 49. In some embodiments, a CAR with a CD70 antigen binding domain is encoded by a nucleic acid that comprises, consists of, or consists essentially of a sequence 75%, 80%, 85%, 90%, 95%, or 100% (and any value therebetween) identical to SEQ ID NO: 51. In some embodiments, a CAR with a CD70 antigen binding domain comprises a truncated CD27 (tCD27) extracellular domain that comprises, consists of, or consists essentially of a sequence 75%, 80%, 85%, 90%, 95%, or 100% (and any value therebetween) identical to SEQ ID NO: 48. In some embodiments, a CAR with a CD70 antigen binding domain comprises a tCD27 extracellular domain encoded by a nucleic acid that comprises, consists of, or consists essentially of a sequence 75%, 80%, 85%, 90%, 95%, or 100% (and any value therebetween) identical to SEQ ID NO: 52.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M. In some embodiments, the KD of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

In some embodiments, an antigen binding domain of a CAR is an Fc region binding domain (e.g., binds an immunoglobulin Fc domain). In certain embodiments, an antigen binding domain of a CAR is derived from an immunoglobulin Fc receptor (FcR). In certain embodiments, an antigen binding domain of a CAR that binds an Fc region may is derived from a CD16 gene sequence. In certain embodiments, an FcR is for IgG (e.g., FcγRI/CD64, FcγRII/CD32, and FcγRIII/CD16), IgE (e.g., FcϵRI), IgA (e.g., FcαRI/CD89), IgM (e.g., FcμR), and/or IgA/IgM (e.g., Fcα/μR). In certain embodiments, an FcR comprises, consists of, or consists essentially of a sequence 75%, 80%, 85%, 90%, 95%, or 100% (and any value therebetween) identical to SEQ ID NO: 9-17. In some embodiments, a CAR with an FcR antigen binding domain is encoded by a nucleic acid that comprises, consists of, or consists essentially of a sequence 75%, 80%, 85%, 90%, 95%, or 100% (and any value therebetween) identical to SEQ ID NO: 40.

In some embodiments, a CAR with an FcR antigen binding domain is utilized in conjunction with an additional immunotherapy, e.g., an antibody based therapy. In some embodiments, a CAR with an FcR antigen binding domain provides a universal CAR that can be used with any antibody therapy, so long as the antibody as an Fc domain that can be targeted. In some embodiments, an FcR CAR can serve as a CAR and also as a transduction marker and/or safety switch.

### 3. Peptide Spacer

A peptide spacer (e.g., a spacer), such as an extracellular spacer may link an antigen-binding domain to a transmembrane domain. In some embodiments, a peptide spacer is flexible enough to allow the antigen-binding domain to orient in different directions to facilitate antigen binding. In some embodiments herein, a peptide spacer is a “hinge”, e.g., it is a flexible polypeptide connector region that connects one or more domains of a CAR to one or more other domains of a CAR. As used herein, the term “hinge” refers to a flexible polypeptide connector region (also referred to herein as “hinge region”) providing structural flexibility and spacing to flanking polypeptide regions and can consist of natural or synthetic polypeptides.

In some embodiments, a peptide spacer comprises, consists essentially of, or consists of a sequence derived from a CD30 gene. In some embodiments, a peptide spacer comprises, consists essentially of, or consists of a sequence derived from a mammalian CD30 gene. In some embodiments, a peptide spacer comprises, consists essentially of, or consists of a sequence derived from a mouse CD30 gene. In some embodiments, a peptide spacer comprises, consists essentially of, or consists of a sequence derived from a human CD30 gene. In some embodiments, a peptide spacer comprises, consists essentially of, or consists of a sequence derived from a human CD30 coding region. In some embodiments, a peptide spacer comprises, consists essentially of, or consists of a sequence derived from a human CD30 transcript isoform 1 coding region.

In some embodiments, a spacer comprises the hinge region from IgG. In some embodiments, a spacer comprises or further comprises the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4. An extracellular spacer may comprise a hinge region.

A “hinge” derived from an immunoglobulin (e.g., IgGl) is generally defined as stretching from Glu216 to Pro230 of human IgGI (Burton (1985) Molec. Immunol., 22:161-206). Hinge regions of other IgG isotypes may be aligned with the IgGI sequence by placing the first and last cysteine residues forming inter-heavy chain disulfide (S-S) bonds in the same positions.

In some embodiments, a hinge region may be of natural occurrence or non-natural occurrence, including but not limited to an altered hinge region as described in U.S. Pat. No. 5,677,425, incorporated by reference herein. In some embodiments, a hinge region can include a complete hinge region derived from an antibody of a different class or subclass from that of the CH1 domain. In some embodiments, the term “hinge” and/or “peptide spacer” can also include regions derived from CD8 and other receptors that provide a similar function in providing flexibility and spacing to flanking regions.

In certain embodiments, a hinge region is derived from CD30. In certain embodiments, a hinge does not comprise a cysteine. In certain embodiments, a hinge is enriched for G and/or S amino acids relative to other hinges known in the art. In certain embodiments, a hinge and a transmembrane domain are derived from the same gene. In some embodiments, a hinge and a transmembrane domain are derived from the same coding sequence. In some embodiments, where an immune cell is designed to express more than one CAR, a unique hinge region is utilized for each CAR, e.g., one CD30 hinge, one CD8 hinge, etc.

In some embodiments, an extracellular peptide spacer comprising a hinge can have a length of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, a peptide spacer is 42 amino acids in length.

In some embodiments, an extracellular peptide spacer comprising a hinge can have a length of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein).

In some embodiments, an extracellular spacer comprises, consists essentially of, or consists of a hinge region from an immunoglobulin (e.g., IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer can have an effect on a CAR's signaling activity, a CARs expression levels (transcription and/or translation), cytotoxicity and/or cancer cell killing efficacy, and/or a CAR cells' expansion properties in response to antigen-stimulated CAR signaling. In some embodiments, a CARs extracellular spacer sequence is dependent on the location of the target antigen. In some embodiments, where a target antigen is proximal to a cell membrane, a longer extracellular spacer is used. In some embodiments, where a target antigen is distal to a cell membrane, a shorter extracellular spacer is used. In some embodiments, where a more flexible CAR is desired, a longer extracellular spacer is used. In some embodiments, where a more rigid CAR is desired, a shorter extracellular spacer is used.

In some embodiments, a shorter spacer such as less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, or 2 amino acids is used. In some embodiments, a shorter spacer may have an advantage in CAR mediated signaling activity, a CARs expression levels (transcription and/or translation), cytotoxicity and/or cancer cell killing efficacy, and/or a CAR cells' expansion properties in response to antigen-stimulated CAR signaling.

In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids is used. In some embodiments, a longer spacer may have an advantage in CAR mediated signaling activity, a CARs expression levels (transcription and/or translation), cytotoxicity and/or cancer cell killing efficacy, and/or a CAR cells' expansion properties in response to antigen-stimulated CAR signaling.

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and other regions, (e.g., the CH2 or CH3 region or between the CH2 and CH3 region when both are present). In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

In certain embodiments, a peptide spacer may come from any suitable source, but in specific embodiments a peptide spacer is from CD30, CD8a, CD28, PD-1, CTLA4, alpha, beta or zeta chain of the T-cell receptor, CD2, CD3 zeta, CD3 epsilon, CD3 gamma, CD3 delta, CD45, CD4, CD5, CD8b, CD9, CD16, CD22, CD27, CD32, CD33, CD37, CD64, CD80, CD86, OX-40 (CD134), CD137, CD154, CD160, BTLA, LAIR1, TIGIT, TIM4, ICOS/CD278, GITR/CD357, NKG2D, LAG-3, PD-L1, PD-1, TIM-3, HVEM, LIGHT, DR3, CD30, CD224, CD244, SLAM, CD226, DAP, or a combination thereof or others. In specific embodiments, a peptide spacer is from CD30. In specific embodiments, a peptide spacer is from an immunoglobulin light chain junction region.

### 4. Transmembrane Domain

Polypeptides of the present disclosure may comprise a transmembrane domain. In some embodiments, a transmembrane domain is a hydrophobic alpha helix that spans the membrane. Different transmembrane domains may result in different receptor stability.

In some embodiments, the transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

In some embodiments, a transmembrane domain comprises, consists essentially of, or consists of a sequence derived from a CD30 gene. In some embodiments, a transmembrane domain comprises, consists essentially of, or consists of a sequence derived from a mammalian CD30 gene. In some embodiments, a transmembrane domain comprises, consists essentially of, or consists of a sequence derived from a mouse CD30 gene. In some embodiments, a transmembrane domain comprises, consists essentially of, or consists of a sequence derived from a human CD30 gene. In some embodiments, a transmembrane domain comprises, consists essentially of, or consists of a sequence derived from a human CD30 coding region. In some embodiments, a transmembrane domain comprises, consists essentially of, or consists of a sequence derived from a human CD30 transcript isoform 1 coding region.

In certain embodiments, a transmembrane domain does not comprise a cysteine. In certain embodiments, a hinge and a transmembrane domain are derived from the same gene. In some embodiments, a hinge and a transmembrane domain are derived from the same coding sequence. In certain embodiments, a hinge and a transmembrane domain are contiguous amino acids derived from a wild type gene. In some embodiments, where an immune cell is designed to express more than one CAR, a unique transmembrane region is utilized for each CAR, e.g., one CD30 transmembrane region, one CD8 transmembrane region, etc.

In some embodiments, a transmembrane domain can have a length of at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, a transmembrane domain is 21 amino acids in length. In some embodiments, a transmembrane domain is not 28, 27, 26, 25, 24, 23, or 22 amino acids in length.

In some embodiments, a transmembrane domain comprises, consists essentially of, or consists of a sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 4.

In certain embodiments, a transmembrane domain lacks the amino acid sequence PVLDAG. In certain embodiments, a transmembrane domain lacks the amino acid sequence VLDAG. In certain embodiments, a transmembrane domain lacks the amino acid sequence LDAG. In certain embodiments, a transmembrane domain lacks the amino acid sequence DAG. In certain embodiments, a transmembrane domain lacks the amino acid sequence AG.

In some embodiments, a transmembrane domain and a peptide spacer comprises, consists essentially of, or consists of a sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1.

In some embodiments, a transmembrane domain and/or a peptide spacer do not comprise 3 or more contiguous amino acids according to SEQ ID NO: 5.

In some embodiments, any transmembrane domain that provides for insertion of a polypeptide into the cell membrane of a eukaryotic (e.g., mammalian) cell may be suitable for use. In some embodiments, a transmembrane domain is derived from CD30, CD28, CD8, CD4, CD3-zeta, OX-40 (CD134), or CD7. In some embodiments, a transmembrane domain is derived the alpha, beta or zeta chain of the T-cell receptor, CD28, CD2, CD3 zeta, CD3 epsilon, CD3 gamma, CD3 delta, CD45, CD4, CD5, CD8 (including CD8alpha), CD9, CD 16, CD22, CD33, CD37, CD64, CD80, CD86, CD 134, OX-40 (CD134), 4-1BB (CD137, CD154, ICOS/CD278, GITR/CD357, NKG2D, PD-1, CTLA4, and DAP molecules. In some embodiments, a transmembrane domain is synthetic. In some aspects, a synthetic transmembrane domain comprises predominantly hydrophobic residues such as leucine and valine. In some aspects, a triplet of phenylalanine, tryptophan and valine will be found at each end of a synthetic transmembrane domain.

### 5. Cytoplasmic Region

After antigen recognition, receptors of the present disclosure may cluster and a signal transmitted to the cell through the cytoplasmic region. In some embodiments, the costimulatory domains described herein are part of the cytoplasmic region. In some embodiments, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2 (L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2 (L/I)) (X3)n (YX1X2 (L/I)), where n is an integer from 6 to 8, and each of the 6-8 X3 can be any amino acid.

In some embodiments, a suitable cytoplasmic region is an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that, in some embodiments, are incorporated in a CAR of the disclosure:

In some embodiments, a suitable cytoplasmic region can comprise an ITAM motif-containing portion of the full length DAP12 amino acid sequence. In some embodiments, the cytoplasmic region is derived from FCER1G (also known as FCRG; Fc epsilon receptor I gamma chain; Fc receptor gamma-chain; fc-epsilon Rl-gamma; fcRgamma; fceRI gamma; high affinity immunoglobulin epsilon receptor subunit gamma; immunoglobulin E receptor, high affinity, gamma chain; etc.). In some embodiments, a suitable cytoplasmic region can comprise an ITAM motif-containing portion of the full length FCER1G amino acid sequence.

In some embodiments, the cytoplasmic region is derived from T cell surface glycoprotein CD3 delta chain (also known as CD3D; CD3-DELTA; T3D; CD3 antigen, delta subunit; CD3 delta; CD38; CD3d antigen, delta polypeptide (TiT3 complex); OKT3, delta chain; T cell receptor T3 delta chain; T cell surface glycoprotein CD3 delta chain; etc.). In some embodiments, a suitable cytoplasmic region can comprise an ITAM motif-containing portion of the full length CD3 delta amino acid sequence. In some embodiments, the cytoplasmic region is derived from T cell surface glycoprotein CD3 epsilon chain (also known as CD3e, CD38; T cell surface antigen T3/Leu-4 epsilon chain, T cell surface glycoprotein CD3 epsilon chain, AI504783, CD3, CD3-epsilon, T3e, etc.). In some embodiments, a suitable cytoplasmic region can comprise an ITAM motif-containing portion of the full length CD3 epsilon amino acid sequence. In some embodiments, the cytoplasmic region is derived from T cell surface glycoprotein CD3 gamma chain (also known as CD3G, CD3γ, T cell receptor T3 gamma chain, CD3-GAMMA, T3G, gamma polypeptide (TiT3 complex), etc.). In some embodiments, a suitable cytoplasmic region can comprise an ITAM motif-containing portion of the full length CD3 gamma amino acid sequence. In some embodiments, the cytoplasmic region is derived from T cell surface glycoprotein CD3 zeta chain (also known as CD3Z, CD35, T cell receptor T3 zeta chain, CD247, CD3-ZETA, CD3H, CD3Q, T3Z, TCRZ, etc.). In some embodiments, a suitable cytoplasmic region can comprise an ITAM motif-containing portion of the full length CD3 zeta amino acid sequence.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some embodiments, a suitable cytoplasmic region can comprise an ITAM motif-containing portion of the full length CD79A amino acid sequence.

### 6. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM. In some embodiments, a costimulatory region is derived from CD8, 4-1BB (CD137), CD27, CD28, CD30, OX-40 (CD134), CD38, CD35, CD45, CD4, CD5, CD9, CD16, CD22, CD33, CD37, CD64, CD80, CD86, or CD154. In some embodiments, a costimulatory domain includes, but are not limited to one or more of CD28, CD27, OX-40 (CD134), ICOS, HVEM, GITR, LIGHT, CD40L, DR3, CD30, SLAM, CD2, CD226 (DNAM-1), MyD88, CD244, TMIGD2, BTNL3, NKG2D, DAP10, DAP12, 4-1BB (CD137), or a synthetic molecule. In some embodiments, in addition to a primary signal initiated by CD32, an additional signal provided by a costimulatory receptor inserted in a CAR is important for full activation of immune cells and could help improve in vivo persistence and the therapeutic success of the cell therapy.

A costimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDw137; ILA; etc.). In some embodiments, the costimulatory region is derived from an intracellular portion of the transmembrane protein CD28 (also known as Tp44). In some embodiments, the costimulatory region is derived from an intracellular portion of the transmembrane protein ICOS (also known as AILIM, CD278, and CVID1). In some embodiments, the costimulatory region is derived from an intracellular portion of the transmembrane protein OX-40 (also known as TNFRSF4, RP5-902P8.3, ACT35, CD134, OX40, TXGP1L). In some embodiments, the costimulatory region is derived from an intracellular portion of the transmembrane protein BTLA (also known as BTLA1 and CD272). In some embodiments, the costimulatory region is derived from an intracellular portion of the transmembrane protein CD27 (also known as S 152, T14, TNFRSF7, and Tp55). In some embodiments, the costimulatory region is derived from an intracellular portion of the transmembrane protein CD30 (also known as TNFRSF8, D1S166E, and Ki-1). In some embodiments, the costimulatory region is derived from an intracellular portion of the transmembrane protein GITR (also known as TNFRSF18, RP5-902P8.2, AITR, CD357, and GITR-D). In some embodiments, the costimulatory region derived from an intracellular portion of the transmembrane protein HVEM (also known as TNFRSF14, RP3-395M20.6, ATAR, CD270, HVEA, HVEM, LIGHTR, and TR2).

In some embodiments, the polypeptides described herein may further comprise a detection peptide. Suitable detection peptides include hemagglutinin (HA; e.g., YPYDVPDYA (SEQ ID NO: 28)) FLAG (e.g., DYKDDDDK (SEQ ID NO: 29)) c-myc (e.g., EQKLISEEDL; SEQ ID NO: 30)) and the like. Other suitable detection peptides are known in the art.

In some aspects, a CAR of the present disclosure comprises a CD28 costimulatory domain. In some aspects, the CD28 costimulatory domain comprises SEQ ID NO: 15. In some aspects, a CAR of the present disclosure comprises a 4-1BB costimulatory domain. In some aspects, the 4-1BB costimulatory domain comprises SEQ ID NO: 25. In some aspects, a CAR of the present disclosure comprises an OX40 costimulatory domain. In some aspects, the OX40 costimulatory domain comprises SEQ ID NO: 26.

### 7. Peptide Linkers

In some embodiments, the polypeptides of the disclosure include peptide linkers (sometimes referred to as a linker). In some embodiments, a peptide linker is used to separate any of the peptide domain/regions described herein. As an example, in some embodiments, a linker is between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art. In certain embodiments, it may be appropriate for one of skill in the art to adjust the length of peptide linkers to optimize the performance of one or more CAR components. Routine optimization of peptide linker length is within the skillset of one of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS)n (SEQ ID NO: 31), (G4S)n and (GGGS)n (SEQ ID NO: 32), where n is an integer of at least one. In some embodiments, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO: 33), GGSGG (SEQ ID NO: 34), GSGSG (SEQ ID NO: 35), GSGGG (SEQ ID NO: 36), GGGSG (SEQ ID NO: 37), or GSSSG (SEQ ID NO: 38).

### II. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. In some embodiments, nucleic acids that encode an epitope to which certain of the polypeptides provided herein are also provided. In some embodiments, nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Vectors

The polypeptides of the disclosure, including CARs of the disclosure, may be delivered to recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In some embodiments, suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

In some embodiments, an immune cell is transduced with a vector encoding one or more antigen-targeting CARs. In some embodiments, CARs may or may not be comprised on or with the same vector. In some embodiments, one or more CARs are expressed from the same vector molecule, such as the same viral vector molecule. In some embodiments, the expression of one or more CARs may or may not be regulated by the same regulatory element(s). In some embodiments, when more than one CAR is comprised on the same vector, the more than one CAR may or may not be expressed as separate polypeptides. In some embodiments, where more than one CAR is comprised on the same vector and the more than one CAR are expressed as separate polypeptides, they are separated on the vector by a 2A element and/or IRES element (or both kinds are used on the same vector once or more than once), for example.

In some embodiments, a CAR expressing vector is a multicistronic (e.g., bicistronic) vector. In some embodiments, a CAR expressing vector is a multicistronic vector expressing both an anti-CD19 CAR and an anti-CD79B CAR separated by a 2A element. In some embodiments, a CAR expressing vector is a multicistronic vector encoding both an anti-CD19 CAR and an anti-CD79A CAR separated by a 2A element. In some embodiments, a CAR expressing vector is a multicistronic vector encoding both an FcR CAR and an anti-CD79B CAR separated by a 2A element. In some embodiments, a CAR expressing vector is a multicistronic vector encoding both an FcR CAR and an anti-CD19 CAR separated by a 2A element. In some embodiments, a CAR expressing vector is a multicistronic vector encoding a CD70 CAR, and one or more additional CAR molecules. In some embodiments, a CAR expressing vector is a multicistronic vector encoding a anti-CD19 CAR, anti-CD79B CAR, anti-CD79A CAR, an FcR CAR, and/or an anti-CD70 CAR (e.g., tCD27-CAR).

In some embodiments, a 2A element is a T2A element. In some embodiments, a 2A element is a P2A element. In some embodiments, a 2A element is an E2A element.

### B. Sequences

Certain nucleotide sequences of the polynucleotides, including polynucleotides expressing chimeric antigen receptors and portions and regions thereof, are provided in Table 2.

TABLE 2
polynucleotide sequences

| | SEQ ID | |
|---|---|---|
| Name | NO: | Sequence |
| CD30 hinge and | 39 | GCCCCCGCTTCTACTTCACCAACCCAGAGCCTT |
| Transmembrane (TM) | | CTAGTAGACAGTCAGGCCTCCAAGACGCTGCCC |
| domain | | ATCCCTACCTCCGCCCCGGTGGCTCTGAGCTCG |
| | | ACCGGCAAGCCCGTGCTGGATGCAGGTCCCGTG |
| | | CTCTTTTGGGTCATTCTGGTGCTGGTGGTGGTC |
| | | GTTGGCTCCTCTGCGTTCCTGCTG |
| Fc receptor-based CAR | 40 | ATGCTGCTGCTGGTGACATCCCTGCTGCTGTGC |
| | | GAGCTGCCCCATCCCGCCTTTCTGCTGATTGGA |
| | | ATGAGGACTGAGGACCTGCCCAAGGCCGTGGTG |
| | | TTCCTGGAGCCCCAGTGGTACCGGGTGCTGGAG |
| | | AAGGACAGCGTGACCCTGAAGTGCCAGGGCGCC |
| | | TATTCCCCTGAGGATAACTCTACACAGTGGTTC |
| | | CACAATGAGTCTCTGATCAGCTCCCAGGCCTCT |
| | | AGCTACTTTATCGACGCAGCAACCGTGGACGAT |
| | | AGCGGAGAGTATCGGTGCCAGACCAACCTGAGC |
| | | ACACTGTCCGATCCTGTGCAGCTGGAGGTGCAC |
| | | ATCGGATGGCTGCTGCTGCAGGCCCCAAGATGG |
| | | GTGTTTAAGGAGGAGGACCCTATCCACCTGAGA |
| | | TGTCACTCCTGGAAGAATACCGCCCTGCACAAG |
| | | GTGACATACCTGCAGAACGGCAAGGGCAGGAAG |
| | | TACTTCCACCACAATAGCGACTTTTATATCCCA |
| | | AAGGCCACCCTGAAGGATTCTGGCAGCTACTTC |
| | | TGCAGGGGCCTGGTGGGCTCCAAGAACGTGTCC |
| | | TCTGAGACAGTGAATATCACCATCACAGTCAGC |
| | | AGTGCACCGGCTTCCACGTCCCCCACCCAAAGC |
| | | TTGCTGGTAGACTCCCAAGCCAGTAAGACACTT |
| | | CCCATACCAACAAGCGCGCCGGTAGCGTTGTCA |
| | | TCCACGGGAAAACCCGTCTTGGACGCAGGGCCA |
| | | GTCCTGTTTTGGGTAATTCTGGTTTTGGTCGTT |
| | | GTGGTTGGCTCAAGTGCGTTCCTCCTGTATTGC |
| | | TGGGTTCGAAGTAAACGCTCAAGGCTTCTTCAC |
| | | TCCGACTATATGAATATGACCCCGAGGAGACCT |
| | | GGGCCAACGCGGAAACATTATCAACCCTATGCC |
| | | CCCCCTAGGGACTTTGCGGCTTATAGGTCCCGC |
| | | GTAAAGTTTAGCCGGAGCGCAGACGCCATCACC |
| | | TCTTACGAGAAGAGCGACGGCGTTTACACGGGG |
| | | CTGTCTACCAGGAATCAAGAGACTTACGAAACC |
| | | CTTAAACATGAAAAACCCCCACAGGCCATCCGA |
| | | AAACGACAGCCCGAAGAAACAAATAATGACTAC |
| | | GAGACCGCCGATGGAGGATATATGACTCTTAAC |
| | | CCTAGGGCTCCTACCGATGACGATAAAAATATC |
| | | TACTTGACACTGCCTCCCAACGACCATGTCAAC |
| | | AGTAACAATTATACTGGGCTGTCAACCCGGAAT |
| | | CAAGAAACGTATGAGACGTTGAAACATGAGAAG |
| | | CCCCCACAA |
| CD19 CAR (FMC63 | 41 | ATGGCCCTGCCAGTGACCGCCCTGCTGCTGCCA |
| SCFV-CD30 HTM-CD28 | | CTGGCACTGCTGCTGCACGCAGCAAGGCCAGAC |
| cos-CD3Zeta) | | ATCCAGATGACACAGACCACAAGCTCCCTGTCC |
| | | GCCTCTCTGGGCGACAGAGTGACCATCTCTTGC |
| | | AGGGCCAGCCAGGATATCTCCAAGTATCTGAAT |
| | | TGGTACCAGCAGAAGCCTGATGGCACAGTGAAG |
| | | CTGCTGATCTATCACACCTCTAGACTGCACAGC |
| | | GGCGTGCCATCCAGGTTTAGCGGCTCCGGCTCT |
| | | GGCACAGACTACTCTCTGACCATCAGCAATCTG |
| | | GAGCAGGAGGATATCGCCACCTATTTCTGCCAG |
| | | CAGGGCAACACACTGCCTTACACCTTTGGCGGC |
| | | GGCACAAAGCTGGAGATCACCGGCGGCGGCGGC |
| | | TCTGGAGGAGGAGGAAGCGGAGGAGGAGGATCC |
| | | GAGGTGAAGCTGCAGGAGAGCGGACCAGGACTG |
| | | GTGGCACCCAGCCAGTCCCTGTCTGTGACATGT |
| | | ACCGTGTCCGGCGTGTCTCTGCCAGACTACGGC |
| | | GTGAGCTGGATCAGACAGCCACCTAGGAAGGGT |
| | | CTGGAGTGGCTAGGTGTGATTTGGGGCTCCGAG |
| | | ACCACATATTACAACAGCGCGCTCAAGTCCCGC |
| | | CTGACCATTATCAAGGACAACTCCAAGTCGCAG |
| | | GTGTTCCTGAAGATGAATTCCTTGCAGACTGAT |
| | | GACACCGCCATCTATTACTGCGCCAAGCATTAC |
| | | TACTACGGCGGTAGTTACGCCATGGATTATTGG |
| | | GGCCAGGGCACCTCCGTGACCGTAAGCTCGGCC |
| | | CCCGCTTCTACTTCACCAACCCAGAGCCTTCTA |
| | | GTAGACAGTCAGGCCTCCAAGACGCTGCCCATC |
| | | CCTACCTCCGCCCCGGTGGCTCTGAGCTCGACC |
| | | GGCAAGCCCGTGCTGGATGCAGGTCCCGTGCTC |
| | | TTTTGGGTCATTCTGGTGCTGGTGGTGGTCGTT |
| | | GGCTCCTCTGCGTTCCTGCTGTATTGTTGGGTG |
| | | AGAAGCAAGAGATCCAGGCTGCTGCACAGCGAC |
| | | TACATGAATATGACACCAAGGAGACCAGGACCA |
| | | ACCAGGAAGCACTATCAGCCTTACGCACCTCCA |
| | | AGGGACTTCGCAGCATATAGGAGCCGTGTGAAA |
| | | TTTAGCCGGTCCGCGGACGCCCCTGCTTATCAG |
| | | CAGGGACAGAACCAGCTGTACAACGAGCTCAAC |
| | | CTGGGTCGCAGGGAGGAGTACGACGTGCTGGAC |
| | | AAGCGCCGCGGCCGCGACCCAGAAATGGGCGGG |
| | | AAGCCCAGGCGCAAGAACCCACAGGAGGGCCTG |
| | | TATAATGAGCTGCAGAAGGACAAGATGGCCGAG |
| | | GCCTACAGCGAGATCGGCATGAAGGGAGAGAGG |
| | | AGAAGGGGCAAGGGACACGATGGCCTGTATCAG |
| | | GGCCTGTCCACAGCCACCAAGGACACCTACGAT |
| | | GCACTGCACATGCAGGCACTGCCACCTAGA |
| CD79B CAR (SN8 SCFV- | 42 | ATGGCCCTGCCAGTGACCGCCCTGCTGCTGCCA |
| CD30 HTM-CD28 cos- | | CTGGCACTGCTGCTGCACGCAGCAAGGCCAGAC |
| CD3Zeta) | | ATTGTGCTGACACAGTCCCCTGCAAGCCTCGCT |
| | | GTATCTCTCGGTCAGAGGGCCACAATATCATGT |
| | | AAGGCGAGTCAATCCGTTGACTATGATGGAGAT |
| | | AGCTTTTTGAACTGGTACCAACAGAAGCCCGGA |
| | | CAGCCACCGAAGCTCTTCATTTACGCAGCATCA |
| | | AATCTTGAGTCTGGAATACCAGCACGGTTTAGC |
| | | GGATCTGGGAGCGGCACAGATTTTACGTTGAAT |
| | | ATACATCCCGTGGAAGAGGAAGACGCAGCAACG |
| | | TACTATTGTCAACAGTCTAATGAGGATCCATTG |
| | | ACGTTCGGAGCAGGCACAGAGCTCGAACTTAAG |
| | | AGGAGCGGAGGAGGAGGCTCCGGTGGAGGTGGA |
| | | TCTGGTGGTGGAGGCAGCGGAGGCGGAGGCTCA |
| | | GAGGTACAACTTCAGCAGTCCGGCGCTGAACTG |
| | | ATGAAGCCAGGGGCGTCCGTTAAAATTTCTTGT |
| | | AAAGCGACAGGGTATACCTTTAGTTCTTACTGG |
| | | ATTGAATGGGTCAAACAAAGACCAGGACATGGA |
| | | CTCGAATGGATTGGGGAGATCTTGCCAGGAGGA |
| | | GGGGACACCAACTACAACGAGATCTTCAAGGGG |
| | | AAGGCGACGTTCACTGCAGATACTAGCAGCAAC |
| | | ACCGCTTATATGCAGCTCAGTTCACTTACGTCA |
| | | GAGGACAGCGCCGTGTATTATTGCACCAGGAGA |
| | | GTTCCTGTTTATTTTGACTATTGGGGTCAGGGA |
| | | ACTTCCGTTACTGTGTCCAGTGCCCCCGCTTCT |
| | | ACTTCACCAACCCAGAGCCTTCTAGTAGACAGT |
| | | CAGGCCTCCAAGACGCTGCCCATCCCTACCTCC |
| | | GCCCCGGTGGCTCTGAGCTCGACCGGCAAGCCC |
| | | GTGCTGGATGCAGGTCCCGTGCTCTTTTGGGTC |
| | | ATTCTGGTGCTGGTGGTGGTCGTTGGCTCCTCT |
| | | GCGTTCCTGCTGTATTGTTGGGTGAGAAGCAAG |
| | | AGATCCAGGCTGCTGCACAGCGACTACATGAAT |
| | | ATGACACCAAGGAGACCAGGACCAACCAGGAAG |
| | | CACTATCAGCCTTACGCACCTCCAAGGGACTTC |
| | | GCAGCATATAGGAGCCGTGTGAAATTTAGCCGG |
| | | TCCGCGGACGCCCCTGCTTATCAGCAGGGACAG |
| | | AACCAGCTGTACAACGAGCTCAACCTGGGTCGC |
| | | AGGGAGGAGTACGACGTGCTGGACAAGCGCCGC |
| | | GGCCGCGACCCAGAAATGGGCGGGAAGCCCAGG |
| | | CGCAAGAACCCACAGGAGGGCCTGTATAATGAG |
| | | CTGCAGAAGGACAAGATGGCCGAGGCCTACAGC |
| | | GAGATCGGCATGAAGGGAGAGAGGAGAAGGGGC |
| | | AAGGGACACGATGGCCTGTATCAGGGCCTGTCC |
| | | ACAGCCACCAAGGACACCTACGATGCACTGCAC |
| | | ATGCAGGCACTGCCACCTAGA |
| CD70 CAR (tCD27-CD30 | 51 | ATGGCAAGGCCACACCCTTGGTGGCTGTGTGTA |
| HTM-CD28 cos-CD3Zeta) | | CTGGGGACTCTTGTAGGACTGTCCGCGACCCCT |
| | | GCCCCGAAGAGCTGTCCCGAACGACATTATTGG |
| | | GCTCAAGGAAAGTTGTGCTGTCAAATGTGCGAA |
| | | CCGGGTACGTTTCTCGTTAAGGATTGTGACCAG |
| | | CATCGGAAAGCTGCCCAATGCGACCCTTGTATC |
| | | CCCGGAGTTTCTTTTTCACCAGATCACCATACG |
| | | AGGCCGCACTGCGAGTCATGCCGGCACTGCAAT |
| | | AGCGGCCTTCTTGTGAGGAACTGCACAATAACC |
| | | GCAAATGCAGAGTGTGCCTGTAGAAATGGTTGG |
| | | CAATGCCGGGATAAAGAATGTACGGAATGTGAC |
| | | CCTCTGCCAGCCCCCGCTTCTACTTCACCAACC |
| | | CAGAGCCTTCTAGTAGACAGTCAGGCCTCCAAG |
| | | ACGCTGCCCATCCCTACCTCCGCCCCGGTGGCT |
| | | CTGAGCTCGACCGGCAAGCCCGTGCTGGATGCA |
| | | GGTCCCGTGCTCTTTTGGGTCATTCTGGTGCTG |
| | | GTGGTGGTCGTTGGCTCCTCTGCGTTCCTGCTG |
| | | TATTGTTGGGTGAGAAGCAAGAGATCCAGGCTG |
| | | CTGCACAGCGACTACATGAATATGACACCAAGG |
| | | AGACCAGGACCAACCAGGAAGCACTATCAGCCT |
| | | TACGCACCTCCAAGGGACTTCGCAGCATATAGG |
| | | AGCCGTGTGAAATTTAGCCGGTCCGCGGACGCC |
| | | CCTGCTTATCAGCAGGGACAGAACCAGCTGTAC |
| | | AACGAGCTCAACCTGGGTCGCAGGGAGGAGTAC |
| | | GACGTGCTGGACAAGCGCCGCGGCCGCGACCCA |
| | | GAAATGGGCGGGAAGCCCAGGCGCAAGAACCCA |
| | | CAGGAGGGCCTGTATAATGAGCTGCAGAAGGAC |
| | | AAGATGGCCGAGGCCTACAGCGAGATCGGCATG |
| | | AAGGGAGAGAGGAGAAGGGGCAAGGGACACGAT |
| | | GGCCTGTATCAGGGCCTGTCCACAGCCACCAAG |
| | | GACACCTACGATGCACTGCACATGCAGGCACTG |
| | | CCACCTAGA |
| tCD27 | 52 | ATGGCAAGGCCACACCCTTGGTGGCTGTGTGTA |
| | | CTGGGGACTCTTGTAGGACTGTCCGCGACCCCT |
| | | GCCCCGAAGAGCTGTCCCGAACGACATTATTGG |
| | | GCTCAAGGAAAGTTGTGCTGTCAAATGTGCGAA |
| | | CCGGGTACGTTTCTCGTTAAGGATTGTGACCAG |
| | | CATCGGAAAGCTGCCCAATGCGACCCTTGTATC |
| | | CCCGGAGTTTCTTTTTCACCAGATCACCATACG |
| | | AGGCCGCACTGCGAGTCATGCCGGCACTGCAAT |
| | | AGCGGCCTTCTTGTGAGGAACTGCACAATAACC |
| | | GCAAATGCAGAGTGTGCCTGTAGAAATGGTTGG |
| | | CAATGCCGGGATAAAGAATGTACGGAATGTGAC |
| | | CCTCTGCCA |

### C. Mutation

Changes can be introduced by mutation into a nucleic acid, thereby leading to changes in the amino acid sequence of a polypeptide that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Certain mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, e.g., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### III. Therapy

In some embodiments, disclosed herein are methods of treatment comprising disclosed compositions, such as compositions comprising a CAR with a CD30-derived hinge and/or transmembrane domain. In some embodiments, methods of treatment are directed to cancer, while in other embodiments. In some embodiments, methods of treatment are directed to pathogenic and/or exogenous drivers of disease.

In some embodiments, the method further comprises administering a cancer therapy to the patient. The cancer therapy may be chosen based on the expression level measurements, alone or in combination with the clinical risk score calculated for the patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the present disclosure provides methods for immunotherapy comprising administering an effective amount of the compositions that comprise the CAR(s), of the present disclosure. In one embodiment, a medical disease or disorder is treated by administration of a CAR-expressing cell population that elicits an immune response. In certain embodiments of the present disclosure, cancer is treated by administration of a CAR immune cell population that elicits an immune response. Provided herein are methods for treating or delaying progression of cancer in an individual comprising administering to the individual an effective amount of an antigen-specific cell therapy. The present methods may be applied for the treatment of immune disorders, solid cancers, and hematologic cancers, as examples. Specifically, the cancer may be a B cell malignancy, such as diffuse large B-cell lymphoma, high-grade B-cell lymphoma, follicular lymphoma, marginal zone lymphoma, mantle cell lymphoma, Burkitt lymphoma, lymphoplasmacytic lymphoma, hairy cell leukemia, and chronic lymphocytic leukemia.

Certain embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In some embodiments of the methods of the present disclosure, activated CD4 and/or CD8 T cells in the individual are characterized by γ-IFN producing CD4 and/or CD8 T cells and/or enhanced cytolytic activity relative to prior to the administration of the combination. γ-IFN may be measured by any means known in the art, including, e.g., intracellular cytokine staining (ICS) involving cell fixation, permeabilization, and staining with an antibody against γ-IFN. Cytolytic activity may be measured by any means known in the art, e.g., using a cell killing assay with mixed effector and target cells.

In some embodiments, the subject can be administered nonmyeloablative lymphodepleting chemotherapy prior to the T cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T cells. Examples of suitable T-cell growth factors include interleukin (IL)-2, IL-7, IL-15, and/or IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

Intratumoral injection, or injection into the tumor vasculature is specifically contemplated for discrete, solid, accessible tumors. Local, regional or systemic administration also may be appropriate. In some embodiments, for tumors of >4 cm, the volume to be administered will be about 4-10 ml (in particular 10 ml), while for tumors of <4 cm, a volume of about 1-3 ml will be used (in particular 3 ml). In some embodiments, multiple injections delivered as single doses can comprise about 0.1 to about 0.5 ml volumes.

In some embodiments, a cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ cells/m². In a certain embodiment, the dose used in the treatment of human subjects ranges from about 3.8×10⁹ to about 3.8×10¹⁰ cells/m². In additional embodiments, a therapeutically effective amount of cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of cells is readily determined by one of skill in the art based on the age, weight, sex, and physiological condition of the subject. Effective doses can be extrapolated from dose-response curves derived from in vitro or animal model test systems.

In certain embodiments of the present disclosure, an effective amount of CAR-expressing immune cells are delivered to an individual in need thereof, such as an individual that has cancer. In some embodiments, cells can then enhance the individual's immune system to attack cancer cells. In some cases, an individual is provided with one or more doses of immune cells (e.g., those described herein). In some embodiments, where the individual is provided with two or more doses of immune cells, the duration between the administrations should be sufficient to allow time for propagation in the individual, and in specific embodiments the duration between doses is 1, 2, 3, 4, 5, 6, 7, or more days.

In specific embodiments, the cells that have been engineered to express a CAR are provided to an individual in a therapeutically effective amount (in a range from 10³ to 10¹⁰) that ameliorates at least one symptom related to cancer cells in the individual. A therapeutically effective amount may be from 10³ to 10¹⁰, 10³ to 10⁹, 10³ to 10⁸, 10³ to 10⁷, 10³ to 10⁶, 10³ to 10⁵, 10³ to 10⁴, 10⁴ to 10¹⁰, 10⁴ to 10⁹, 10⁴ to 10⁸, 10⁴ to 10⁷, 10⁴ to 10⁶, 10⁴ to 10⁵, 10⁵ to 10¹⁰, 10⁵ to 10⁹, 10⁵ to 10⁸, 10⁵ to 10⁷, 10⁵ to 10⁶, 10⁶ to 10¹⁰, 10⁶ to 10⁹, 10⁶ to 10⁸, 10⁶ to 10⁷, 10⁷ to 10¹⁰, 10⁷ to 10⁹, 10⁷ to 10⁸, 10⁸ to 10¹⁰, 10⁸ to 10⁹, or 10⁹ to 10¹⁰ cells. Thus, in particular embodiments an individual having a certain cancer is provided once or multiple times a therapeutically effective amount of cells expressing one or more CARs.

### A. Cancer Antigens

Among the cancer antigens targeted by CARs of the present disclosure, are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, any suitable antigen may find use in the context of the present disclosure. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, CD20, CD22, CD30, CD70, CD79a, CD79b, SLAM-F7NY-ESO, EGFRVIII, Muc-1, Her2, CA-125, WT-1, Mage-A3, Mage-A4, Mage-A10, TRAIL/DR4, CEA. In particular aspects, the antigens for the one or two or more antigen receptors include, but are not limited to, CD19, EBNA, WT1, CD123, NY-ESO, EGFRVIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRVIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

In some embodiments, tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

In some embodiments, tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers

In some embodiments, tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, bORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

In some embodiments, antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### B. Non-Cancer Antigens

In certain embodiments, an antigen may be microbial. In some embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, coronaviruses such as SARS-COV, SARS-COV-2, or MERS, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### IV. Cellular Therapies

Certain embodiments relate to cells comprising polypeptides or nucleic acids of the disclosure. In some embodiments the cell is an immune cell. In certain embodiments, as used herein, “T cell” includes all types of immune cells expressing CD3 including T-helper cells, invariant natural killer T (INKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### A. Infinite Immune Cells

Certain embodiments of the present disclosure concern immune cells that are engineered to express one or more genes. The expression of the one or more genes directly or indirectly results in the increased lifespan of the cells compared to cells that lack the expression of the one or more genes. In particular embodiments, the cells are manipulated to express the one or more genes, including one or more heterologous genes. In other cases, the cells are manipulated to have upregulation of expression of the one or more genes that are endogenous to the cells, such as through manipulation of one or more regulatory elements of the one or more endogenous genes to the cells. In certain embodiments, methods and compositions related to infinite immune cells are described in PCT Patent Application Publication No. WO/2021/034982, incorporated herein by reference in its entirety.

In particular embodiments, immune cells are manipulated to express BCL6 and one or more pro-survival genes or anti-apoptotic genes or cell survival-promoting genes (and there may or may not be overlap in a gene that is classified as pro-survival or anti-apoptotic or cell survival-promoting). As used herein, the pro-survival gene refers to a nucleic acid polymer that can exert anti-apoptosis function or promote survival by any mechanism. The nucleic acid polymer that can exert anti-apoptosis function may be one or more of Bcl2 family genes such as BCL-XL, BCL-2, MCL-1, Bcl-w, Bfl-1, BCL-B, etc. The nucleic acid polymer that can exert anti-apoptosis function may be one or more of inhibitor of apoptosis (IAP) family genes, such as XIAP, c-IAPl, C-IAP2, NAIP, and Survivin, etc. The nucleic acid polymer that can exert anti-apoptosis function may be able to inhibit or knock out expression of one or more caspases that play a role in apoptosis, such as Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14. Nucleic acid polymers for knockdown or knock-out could be an shRNA expression cassette, or these caspase genes can also be knocked out by gene editing method (CRISPR, TALEN, Zinc finger method, etc.). The nucleic acid polymer that can exert anti-apoptosis function may be able to inhibit or knock out expression of one or more pro-apoptotic genes, such as BIM, Puma, Noxa, Bik, Bmf, Bad, Hrk, Bid, BAX, BAK, BOK, etc. The nucleic acid polymer that can exert anti-apoptosis function may have an anti-apoptotic effect, such as insulin-like growth factor (IGF-1), Hsp70, Hsp27, cFLIP, BNIP3, FADD, Akt, and NF-κB, Raf-1 and MEK1, p90Rsk, C-Jun, BNIP2, BAG1, HSPA9, HSP90B1, miRNA21, miR-106b-25, miR-206, miR-221/222, miR-17-92, miR-133, miR-143, miR-145, miR-155, miR-330, etc.

Infinite T cells may be generated with either wild type or mutant BCL6. The inventors determined that infinite T cells could be generated with either wildtype BCL6 or mutant BCL6 with a single particular nucleotide difference—the codon of the amino acid at position 395 in wild type BCL6 is CCT (encoding Proline/P) and the codon of the amino acid at position 395 in mutant BCL6 is CTT (encoding Leucine/L). The nucleotide and amino acid sequences for the two BCL6 genes are shown below (with the point of mutation in the wildtype sequence being underlined).

SEQ ID NO: 43-Amino Acid sequence of wildtype BCL6:
(SEQ ID NO: 43)
MASPADSCIQFTRHASDVLLNLNRLRSRDILTDVVIVVSREQFRAHKTVLMACSGLFYSIFT
DQLKCNLSVINLDPEINPEGFCILLDFMYTSRLNLREGNIMAVMATAMYLQMEHVVDTCRKF
IKASEAEMVSAIKPPREEFLNSRMLMPQDIMAYRGREVVENNLPLRSAPGCESRAFAPSLYS
GLSTPPASYSMYSHLPVSSLLFSDEEFRDVRMPVANPFPKERALPCDSARPVPGEYSRPTLE
VSPNVCHSNIYSPKETIPEEARSDMHYSVAEGLKPAAPSARNAPYFPCDKASKEEERPSSED
EIALHFEPPNAPLNRKGLVSPQSPQKSDCQPNSPTESCSSKNACILQASGSPPAKSPTDPKA
CNWKKYKFIVLNSLNQNAKPEGPEQAELGRLSPRAYTAPPACQPPMEPENLDLQSPTKLSAS
GEDSTIPQASRLNNIVNRSMTGSPRSSSESHSPLYMHPPKCTSCGSQSPQHAEMCLHTAGPT
FPEEMGETQSEYSDSSCENGAFFCNECDCRFSEEASLKRHTLQTHSDKPYKCDRCQASFRYK
GNLASHKTVHTGEKPYRCNICGAQFNRPANLKTHTRIHSGEKPYKCETCGARFVQVAHLRAH
VLIHTGEKPYPCEICGTRFRHLQTLKSHLRIHTGEKPYHCEKCNLHFRHKSQLRLHLROKHG
AITNTKVOYRVSATDLPPELPKAC
SEQ ID NO: 44-nucleotide sequence of wildtype BCL6 (with the codon
for the point of mutation in the wildtype sequence being
underlined):
(SEQ ID NO: 44)
ATGgcctcgccggctgacagctgtatccagttcacccgccatgccagtgatgttcttctcaa
ccttaatcgtctccggagtcgagacatcttgactgatgttgtcattgttgtgagccgtgagc
agtttagagcccataaaacggtcctcatggcctgcagtggcctgttctatagcatctttaca
gaccagttgaaatgcaaccttagtgtgatcaatctagatcctgagatcaaccctgagggatt
ctgcatcctcctggacttcatgtacacatctcggctcaatttgcgggagggcaacatcatgg
ctgtgatggccacggctatgtacctgcagatggagcatgttgtggacacttgccggaagttt
attaaggccagtgaagcagagatggtttctgccatcaagcctcctcgtgaagagttcctcaa
cagccggatgctgatgccccaagacatcatggcctatcggggtcgtgaggtggtggagaaca
acctgccactgaggagcgcccctgggtgtgagagcagagcctttgcccccagcctgtacagt
ggcctgtccacaccgccagcctcttattccatgtacagccacctccctgtcagcagcctcct
cttctccgatgaggagtttcgggatgtccggatgcctgtggccaaccccttccccaaggagc
gggcactcccatgtgatagtgccaggccagtccctggtgagtacagccggccgactttggag
gtgtcccccaatgtgtgccacagcaatatctattcacccaaggaaacaatcccagaagaggc
acgaagtgatatgcactacagtgtggctgagggcctcaaacctgctgccccctcagcccgaa
atgccccctacttcccttgtgacaaggccagcaaagaagaagagagaccctcctcggaagat
gagattgccctgcatttcgagccccccaatgcacccctgaaccggaagggtctggttagtcc
acagagcccccagaaatctgactgccagcccaactcgcccacagagtcctgcagcagtaaga
atgcctgcatcctccaggcttctggctcccctccagccaagagccccactgaccccaaagcc
tgcaactggaagaaatacaagttcatcgtgctcaacagcctcaaccagaatgccaaaccaga
ggggcCtgagcaggctgagctgggccgcctttccccacgagcctacacggccccacctgcct
gccagccacccatggagcctgagaaccttgacctccagtccccaaccaagctgagtgccagc
ggggaggactccaccatcccacaagccagccggctcaataacatcgttaacaggtccatgac
gggctctccccgcagcagcagcgagagccactcaccactctacatgcaccccccgaagtgca
cgtcctgcggctctcagtccccacagcatgcagagatgtgcctccacaccgctggccccacg
ttccctgaggagatgggagagacccagtctgagtactcagattctagctgtgagaacggggc
cttcttctgcaatgagtgtgactgccgcttctctgaggaggcctcactcaagaggcacacgc
tgcagacccacagtgacaaaccctacaagtgtgaccgctgccaggcctccttccgctacaag
ggcaacctcgccagccacaagaccgtccataccggtgagaaaccctatcgttgcaacatctg
tggggcccagttcaaccggccagccaacctgaaaacccacactcgaattcactctggagaga
agccctacaaatgcgaaacctgcggagccagatttgtacaggtggcccacctccgtgcccat
gtgcttatccacactggtgagaagccctatccctgtgaaatctgtggcacccgtttccggca
ccttcagactctgaagagccacctgcgaatccacacaggagagaaaccttaccattgtgaga
agtgtaacctgcatttccgtcacaaaagccagctgcgacttcacttgcgccagaagcatggc
gccatcaccaacaccaaggtgcaataccgcgtgtcagccactgacctgcctccggagctccc
caaagcctgc
SEQ ID NO: 45-Amino Acid sequence of mutant BCL6 (the leucine
mutation is underlined):
(SEQ ID NO: 45)
MASPADSCIQFTRHASDVLLNLNRLRSRDILTDVVIVVSREQFRAHKTVLMACSGLFYSIFT
DQLKCNLSVINLDPEINPEGFCILLDFMYTSRLNLREGNIMAVMATAMYLQMEHVVDTCRKF
IKASEAEMVSAIKPPREEFLNSRMLMPQDIMAYRGREVVENNLPLRSAPGCESRAFAPSLYS
GLSTPPASYSMYSHLPVSSLLFSDEEFRDVRMPVANPFPKERALPCDSARPVPGEYSRPTLE
VSPNVCHSNIYSPKETIPEEARSDMHYSVAEGLKPAAPSARNAPYFPCDKASKEEERPSSED
EIALHFEPPNAPLNRKGLVSPQSPQKSDCOPNSPTESCSSKNACILQASGSPPAKSPTDPKA
CNWKKYKFIVLNSLNQNAKPEGLEQAELGRLSPRAYTAPPACOPPMEPENLDLQSPTKLSAS
GEDSTIPQASRLNNIVNRSMTGSPRSSSESHSPLYMHPPKCTSCGSQSPQHAEMCLHTAGPT
FPEEMGETQSEYSDSSCENGAFFCNECDCRFSEEASLKRHTLQTHSDKPYKCDRCQASFRYK
GNLASHKTVHTGEKPYRCNICGAQFNRPANLKTHTRIHSGEKPYKCETCGARFVQVAHLRAH
VLIHTGEKPYPCEICGTRFRHLQTLKSHLRIHTGEKPYHCEKCNLHFRHKSQLRLHLROKHG
AITNTKVQYRVSATDLPPELPKAC
SEQ ID NO: 46-Nucleotide sequence of mutant BCL6 (the codon for
leucine is underlined):
(SEQ ID NO: 46)
ATGgcctcgccggctgacagctgtatccagttcacccgccatgccagtgatgttcttctcaa
ccttaatcgtctccggagtcgagacatcttgactgatgttgtcattgttgtgagccgtgagc
agtttagagcccataaaacggtcctcatggcctgcagtggcctgttctatagcatctttaca
gaccagttgaaatgcaaccttagtgtgatcaatctagatcctgagatcaaccctgagggatt
ctgcatcctcctggacttcatgtacacatctcggctcaatttgcgggagggcaacatcatgg
ctgtgatggccacggctatgtacctgcagatggagcatgttgtggacacttgccggaagttt
attaaggccagtgaagcagagatggtttctgccatcaagcctcctcgtgaagagttcctcaa
cagccggatgctgatgccccaagacatcatggcctatcggggtcgtgaggtggtggagaaca
acctgccactgaggagcgcccctgggtgtgagagcagagcctttgcccccagcctgtacagt
ggcctgtccacaccgccagcctcttattccatgtacagccacctccctgtcagcagcctcct
cttctccgatgaggagtttcgggatgtccggatgcctgtggccaaccccttccccaaggagc
gggcactcccatgtgatagtgccaggccagtccctggtgagtacagccggccgactttggag
gtgtcccccaatgtgtgccacagcaatatctattcacccaaggaaacaatcccagaagaggc
acgaagtgatatgcactacagtgtggctgagggcctcaaacctgctgccccctcagcccgaa
atgccccctacttcccttgtgacaaggccagcaaagaagaagagagaccctcctcggaagat
gagattgccctgcatttcgagccccccaatgcacccctgaaccggaagggtctggttagtcc
acagagcccccagaaatctgactgccagcccaactcgcccacagagtcctgcagcagtaaga
atgcctgcatcctccaggcttctggctcccctccagccaagagccccactgaccccaaagcc
tgcaactggaagaaatacaagttcatcgtgctcaacagcctcaaccagaatgccaaaccaga
ggggcTtgagcaggctgagctgggccgcctttccccacgagcctacacggccccacctgcct
gccagccacccatggagcctgagaaccttgacctccagtccccaaccaagctgagtgccagc
ggggaggactccaccatcccacaagccagccggctcaataacatcgttaacaggtccatgac
gggctctccccgcagcagcagcgagagccactcaccactctacatgcaccccccgaagtgca
cgtcctgcggctctcagtccccacagcatgcagagatgtgcctccacaccgctggccccacg
ttccctgaggagatgggagagacccagtctgagtactcagattctagctgtgagaacggggc
cttcttctgcaatgagtgtgactgccgcttctctgaggaggcctcactcaagaggcacacgc
tgcagacccacagtgacaaaccctacaagtgtgaccgctgccaggcctccttccgctacaag
ggcaacctcgccagccacaagaccgtccataccggtgagaaaccctatcgttgcaacatctg
tggggcccagttcaaccggccagccaacctgaaaacccacactcgaattcactctggagaga
agccctacaaatgcgaaacctgcggagccagatttgtacaggtggcccacctccgtgcccat
gtgcttatccacactggtgagaagccctatccctgtgaaatctgtggcacccgtttccggca
ccttcagactctgaagagccacctgcgaatccacacaggagagaaaccttaccattgtgaga
agtgtaacctgcatttccgtcacaaaagccagctgcgacttcacttgcgccagaagcatggc
gccatcaccaacaccaaggtgcaataccgcgtgtcagccactgacctgcctccggagctccc
caaagcctgc

The immune cells may be any kind of immune cells, including T cells (e.g., regulatory T cells, CD4⁺ T cells, CD8⁺ T cells, alpha beta T cells, gamma-delta T cells, or a mixture thereof), NK cells, invariant NKT cells, NKT cells, innate lymphoid cells, or a mixture thereof. The immune cells may be virus-specific, express a CAR, and/or express a TCR. In some embodiments, the cells are monocytes or granulocytes, e.g., myeloid cells, macrophages, neutrophils, dendritic cells (DCs), mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells. These immune cells may be used for therapy as a single cell type or as a combination of multiple immune cell types. In specific embodiments, the immune cells are CD3+, CD4+, CD8+, CD16+, or a mixture thereof.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4- or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, such as a partially or fully histocompatibility matched donor or fully histocompatibility mismatched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor may be allogeneic, provided the cells obtained are subject-compatible in that they can be introduced into the subject. Allogeneic donor cells are may or may not be human-leukocyte-antigen (HLA)-compatible.

Additional methods and compositions related to infinite immune cells are described in PCT Patent Application Publication No. WO/2021/034982, incorporated herein by reference in its entirety.

### 1. T Cells

In some embodiments, the immune cells are T cells. Several basic approaches for the derivation, activation and expansion of functional anti-tumor effector cells have been described in the last two decades. These include: autologous cells, such as tumor-infiltrating lymphocytes (TILs); T cells activated ex-vivo using autologous DCs or PBMCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. These approaches have given rise to numerous protocols for T cell preparation and immunization which can be used in the methods described herein.

In some embodiments, the T cells are derived from the blood, bone marrow, lymph, umbilical cord, or lymphoid organs. In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and gamma/delta T cells. In certain embodiments, T cells are gamma/delta T cells.

In some embodiments, one or more of the T cell populations is enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells or stem cell memory cells is carried out to increase efficacy, such as to improve long-term survival, expansion, and/or engraftment following administration, which in some aspects is particularly robust in such sub-populations.

In some embodiments, the T cells are autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2) or other growth factors.

The cultured T cells can be pooled and rapidly expanded. Rapid expansion provides an increase in the number of antigen-specific T-cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as an human leukocyte antigen A2 (HLA-A2) binding peptide or peptides binding to other MHC class I or class II molecules, in the presence of a T-cell growth factor, such as 300 IU/ml IL-2 or IL-15, with IL-2 being preferred. The in vitro-induced T-cells are rapidly expanded by re-stimulation with the same antigen(s) of the cancer pulsed onto HLA-A2-expressing antigen-presenting cells or antigen-presenting cells expressing other HLA molecules. The in vitro-induced T-cells may also be expanded in the absence of antigen-presenting cells.

The autologous T cells can be modified to express a T cell growth or differentiation factor that promotes the growth, differentiation, and activation of the autologous T cells. Suitable T cell growth factors include, for example, interleukin (IL)-2, IL-7, IL-15, IL-18, IL-21, and IL-12. Suitable methods of modification are known in the art. See, for instance, Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994. In particular aspects, modified autologous T cells express the T cell growth factor at high levels. T cell growth factor coding sequences, such as that of IL-12, are readily available in the art, as are promoters, the operable linkage of which to a T cell growth factor coding sequence promote high-level expression.

### 2. NK Cells

In some embodiments, the immune cells are natural killer (NK) cells. NK cells are a subpopulation of lymphocytes that have spontaneous cytotoxicity against a variety of tumor cells, virus-infected cells, and some normal cells in the bone marrow and thymus. NK cells differentiate and mature in the bone marrow, lymph nodes, spleen, tonsils, and thymus. NK cells can be detected by specific surface markers, such as CD16, CD56, and/or CD8 in humans. NK cells do not express T cell antigen receptors, the pan T marker CD3, or surface immunoglobulin B cell receptors.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, tissues, or umbilical cord blood by methods well known in the art.

### 3. NKT Cells

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids. They constitute only approximately 0.1% of all peripheral blood T cells. NKT cells are a subset of T cells that coexpress an αß T-cell receptor, but also express a variety of molecular markers that are typically associated with NK cells, such as NK1.1. Invariant natural killer T (iNKT) cells express high levels of and are dependent on the transcriptional regulator promyelocytic leukemia zinc finger for their development. Currently, there are five major distinct iNKT cell subsets. These subset cells produce a different set of cytokines once activated. The subtypes iNKT1, iNKT2 and iNKT17 mirror Th cell subsets in cytokine production. In addition, there are subtypes specialized in T follicular helper-like function and IL-10 dependent regulatory functions.

### 4. Innate Lymphoid Cells

Innate lymphoid cells (ILCs) are a group of innate immune cells that are derived from common lymphoid progenitor (CLP) and belong to the lymphoid lineage. These cells are defined by absence of antigen specific B or T cell receptor because of the lack of recombination activating gene (RAG). ILCs do not express myeloid or dendritic cell markers. They play a role in protective immunity and the regulation of homeostasis and inflammation, so their dysregulation can lead to immune pathology such as allergy, bronchial asthma and autoimmune disease. ILCs can be divided based on the cytokines that they can produce, and the transcription factors that regulate their development and function.

### B. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (aMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### C. Cell Generation

Certain methods of the disclosure concern culturing the cells obtained from human tissue samples. In particular embodiments of the present disclosure, cells are plated onto a substrate that allows for adherence of cells thereto. This may be carried out, for example, by plating the cells in a culture plate that displays one or more substrate surfaces compatible with cell adhesion. When the one or more substrate surfaces contact the suspension of cells (e.g., suspension in a medium) introduced into the culture system, cell adhesion between the cells and the substrate surfaces may ensue. Accordingly, in certain embodiments cells are introduced into a culture system that features at least one substrate surface that is generally compatible with adherence of cells thereto, such that the plated cells can contact the said substrate surface, such embodiments encompass plating onto a substrate, which allows adherence of cells thereto.

Cells of the present disclosure may be identified and characterized by their expression of specific marker proteins, such as cell-surface markers. Detection and isolation of these cells can be achieved, for example, through flow cytometry, ELISA, and/or magnetic beads. Reverse-transcription polymerase chain reaction (RT-PCR) may be used to quantify cell-specific genes and/or to monitor changes in gene expression in response to differentiation. In certain embodiments, the marker proteins used to identify and characterize the cells are selected from the list consisting of c-Kit, Nanog, Sox2, Heyl, SMA, Vimentin, Cyclin D2, Snail, E-cadherin, Nkx2.5, GATA4, CD105, CD90, CD29, CD73, Wt1, CD34, CD45, and a combination thereof.

### D. Pharmaceutical Compositions

In certain aspects, the compositions or agents for use in the methods, such as cell based therapy, are suitably contained in a pharmaceutically acceptable carrier. The carrier is non-toxic, biocompatible and is selected so as not to detrimentally affect the biological activity of the agent. The agents in some aspects of the disclosure may be formulated into preparations for local delivery (i.e. to a specific location of the body, such as at a tumor site, or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

Solutions of pharmaceutical compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

In certain aspects, the pharmaceutical compositions are advantageously administered in the form of injectable compositions either as liquid solutions or suspensions; solid forms suitable or solution in, or suspension in, liquid prior to injection may also be prepared. These preparations also may be emulsified. A typical composition for such purpose comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol may be between about 0.01 ml and 0.5 ml, for example.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

### V. Formulations and Culture of the Cells

In particular embodiments, cells of the disclosure may be specifically formulated and/or they may be cultured in a particular medium. Cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, aMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous TCR, which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FR, GD2, G250/CAIX, GD3, Glypican-3 (GPC3), Her2, IL-13Ra2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-BRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### VI. Administration of Compositions

Methods of treatment described herein may comprise monotherapy, or administration of a combination of therapeutic agents, such as a first cancer therapy (e.g., a cell therapy) and a second cancer therapy (e.g., a general pharmaceutical composition). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, pharmaceutical compositions are administered to a subject. Different aspects may involve administering an effective amount of a composition to a subject. In some embodiments, a cellular therapy (e.g., immune cells comprising one or more CARs) is administered to the subject to protect against or treat a condition (e.g., cancer). Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

In some embodiments, immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, monoclonal antibodies such as rituximab, trastuzumab, etc, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

In some embodiments, a first cancer therapy and a second cancer therapy are administered substantially simultaneously. In some embodiments, a first cancer therapy and a second cancer therapy are administered sequentially. In some embodiments, a first cancer therapy, a second cancer therapy, and a third therapy are administered sequentially. In some embodiments, a first cancer therapy is administered before administering a second cancer therapy. In some embodiments, a first cancer therapy is administered after administering a second cancer therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegal1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that additional immunotherapies may be used in combination or in conjunction with methods and compositions of the disclosure. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells, NKT cells, innate lymphoid cells, and NK cells

Antibody-drug conjugates (ADCs) comprise monoclonal antibodies (MAbs) that are covalently linked to cell-killing drugs and may be used in combination therapies. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. Exemplary ADC drugs include ADCETRIS® (brentuximab vedotin) and KADCYLA® (trastuzumab emtansine or T-DM1).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies include immune adjuvants, e.g., Mycobacterium bovis, Plasmodium falciparum, dinitrochlorobenzene, and aromatic compounds); cytokine therapy, e.g., interferons α, β, and γ, IL-1, GM-CSF, and TNF; gene therapy, e.g., TNF, IL-1, IL-2, and p53; and monoclonal antibodies, e.g., anti-CD20, anti-ganglioside GM2, and anti-p185. It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies. Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody that may be used. Pembrolizumab, also known as MK-3475, Merck 3475, lambrolizumab, KEYTRUDA®, and SCH-900475, is an exemplary anti-PD-1 antibody. CT-011, also known as hBAT or hBAT-1, is also an anti-PD-1 antibody. AMP-224, also known as B7-DCIg, is a PDL2-Fc fusion soluble receptor.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off”′ switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. An exemplary anti-CTLA-4 antibody is ipilimumab (also known as 10D1, MDX-010, MDX-101, and Yervoy®) or antigen binding fragments and variants thereof. In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Articles of Manufacture or Kits

An article of manufacture or a kit is provided comprising compositions or methods of using compositions provided herein. For example, a kit comprising one or more immune cells comprising a CAR comprising a CD30-derived hinge and/or transmembrane domain is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Creation of Vectors Comprising Various Hinge Domain, Transmembrane Domain, and/or Costimulatory Domain Combinations

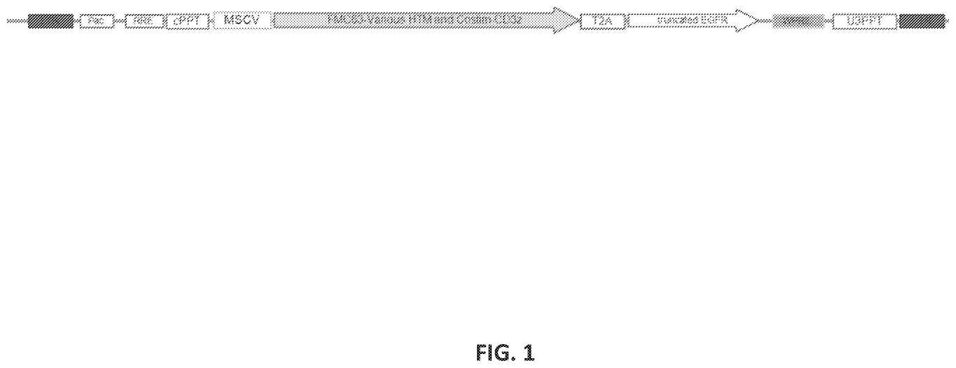

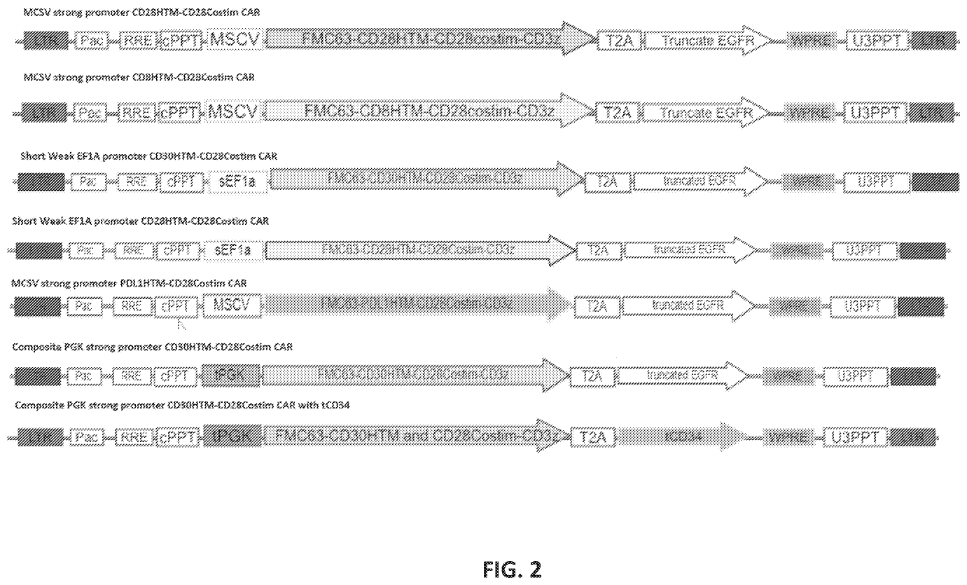

DNA fragments containing different hinge, transmembrane, and/or costimulatory domain combinations were synthesized as Gblocks and cloned between the FMC63 scFv and the CD3ζ (CD3 zeta) domain to build different CARs, using Gibson assembly methods (see FIG. 1). The plasmids expressing different CARs were transfected into 293T cells using lipofectamine 3000 for screening of CARs with acceptable CD19 antigen binding ability. FIG. 2 depicts a subset of exemplary lentiviral vectors comprising CD19-targeting CARs that were used to transduce T cells (e.g., infinite γδ T cells) generated from healthy donor T cells. Vectors were designed following the general scheme as shown in FIG. 2, and were driven by various promoters, such as MSCV, sEF1a=short (weak) EF1alpha promoter, or tPGK=TetO-PGK promoter. The inventors tested over 50 different combinations of various promoter, hinge, transmembrane domain, and costimulatory domains (subset of data shown, additional data available upon request).

### Example 2—Determining Vector Expression in Transduced Cells

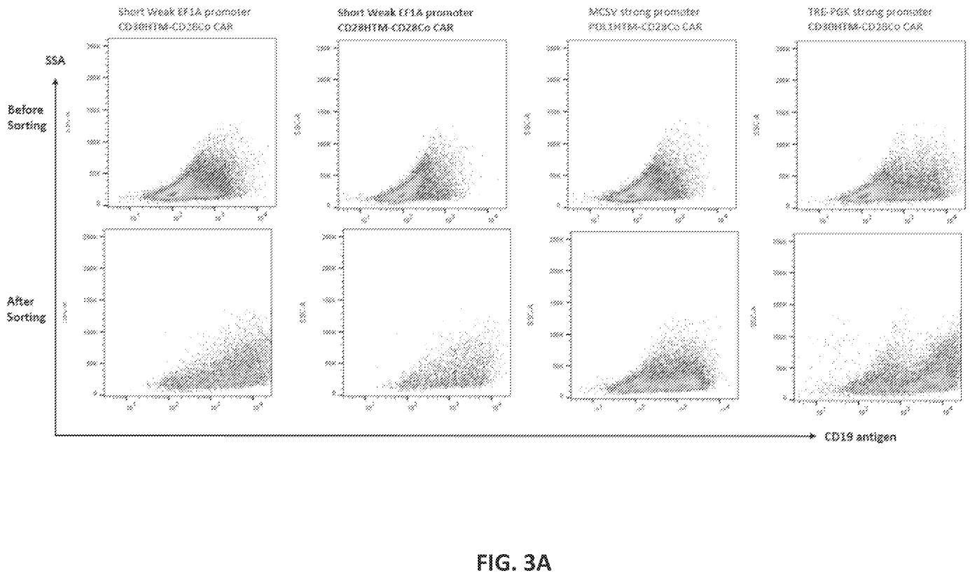

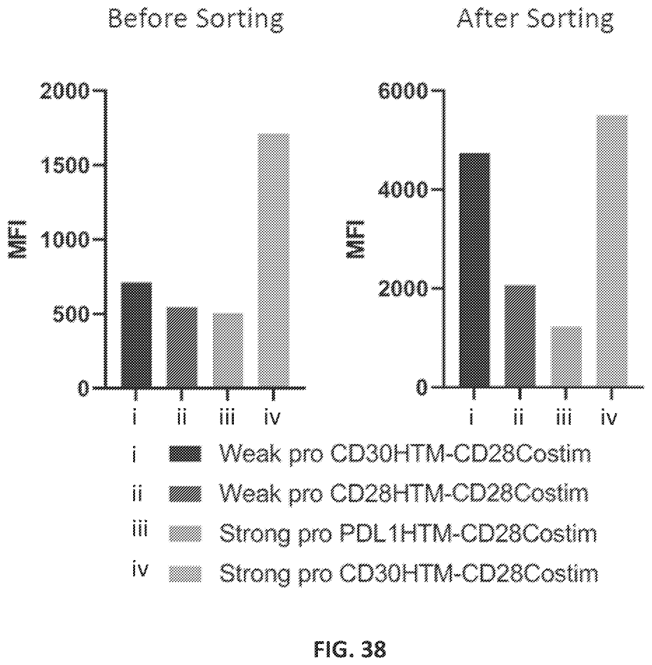

The expression of anti-CD19 CARs with different hinge and transmembrane domains was determined using T cells. In brief, T cells (e.g., infinite γδ T cells) cells were generated from healthy donor T cells by transducing with lentiviral vectors expressing BCL6 and BCL2L1. After expanding the BCL6 and BCL2L1 transduced cells for 4-6 weeks, they were transduced with lentiviral vectors expressing anti-CD19 CARs, with hinge (h), transmembrane (TM), and costimulatory (costim) domains structured as follows: CD28 hTM-CD28 costim, PDL1hTM-CD28 costim, and CD30hTM-CD28 costim. The CAR expression cassettes were driven by a weak (short EF1a promoter) or a strong promoter (the PDL1 hTM CAR used the MSCV promoter, the CD30 hTM CAR used a composite PGK promoter). Anti-CD19 CAR expression was determined by staining with FITC-conjugated CD19 antigen. All the cells were sorted using the FITC conjugated CD19 antigen. The flow cytometry data was analyzed using FlowJo software. All constructs showed clear CAR positive populations before and after sorting, but the CD30 hTMCD28 costim CAR with TRE-PGK promoter showed the highest CD19 CAR expression (see FIGS. 3A-3B). Expression was quantified and the median fluorescence intensity of CAR positive populations from cells transduced was determined (see FIGS. 3A-3B). For the two weak promoter-driven CARs, the MFI of CD30 hTM-CD28 costim CAR population was higher than the CD28 hTM-CD28 costim CAR before and after sorting. For the two strong promoter-driven CARs, the MFI of CD30 hTM-CD28 costim CAR population was higher than the PDL1 hTM-CD28 costim CAR before and after sorting.

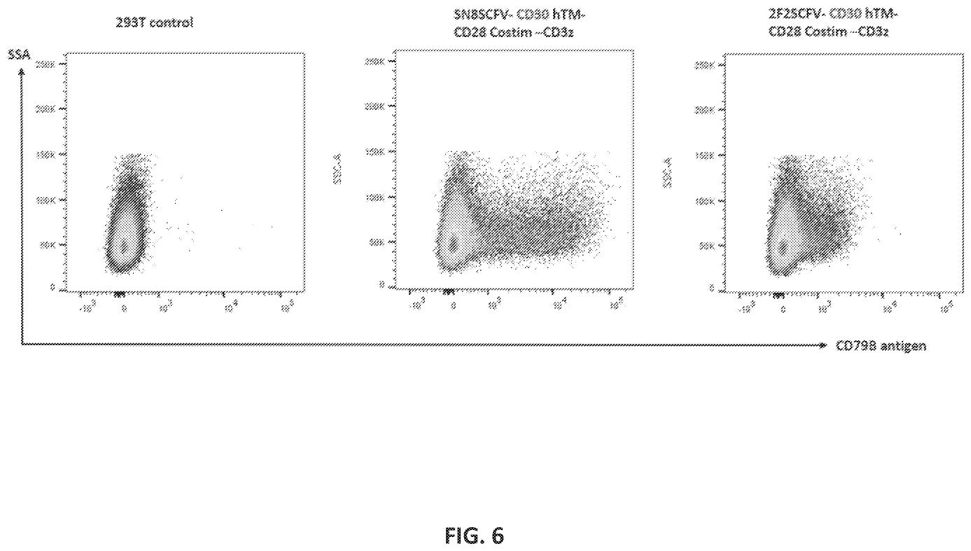

As shown in FIG. 6, expression of anti-CD79B CARs with CD30 hinge and transmembrane domains was determined using 293 T cells. 293T cells were transfected with lentiviral plasmid expressing CD30 hTM-CD28 costim anti-CD79B CARs-one contains an scFv from SN8 clone of CD79B antibody, the other contains an scFv from the 2F2 clone of CD79B antibody. The CAR expression cassettes were driven by the composite human PGK promoter. Twenty-four hours after transfection, anti-79B CAR expression was determined by staining with APC conjugated CD79B antigen. The flow cytometry data was analyzed using FlowJo software. The results showed that the CD30 hinge and transmembrane domains also worked well with scFvs derived from non-CD19 targeting antibodies.

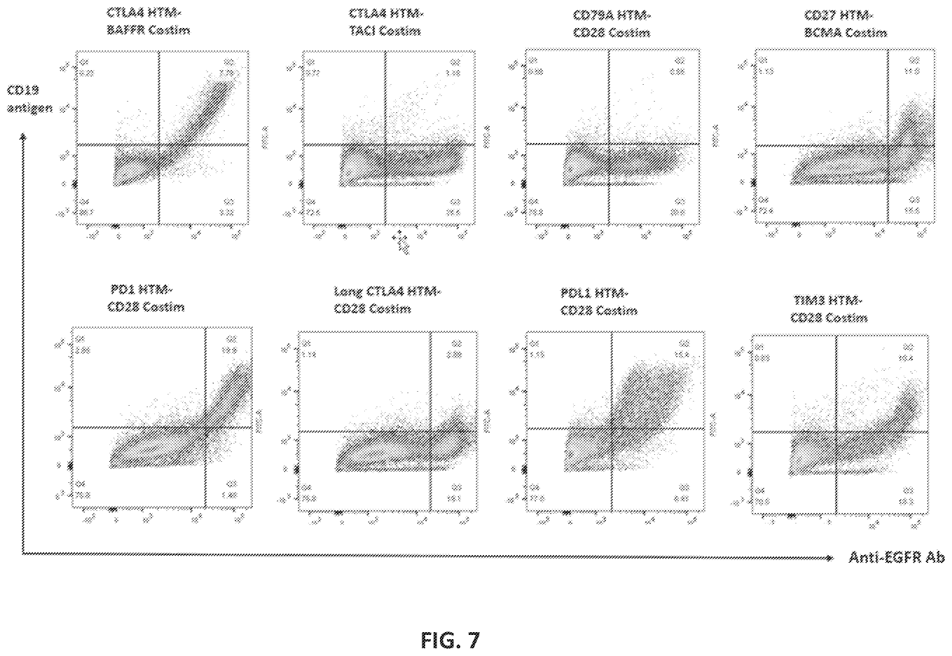

As shown in FIG. 7, expression levels of anti-CD19 CARs with different hinge and transmembrane domains were determined using 293T cells. 293T cells were transfected with lentiviral plasmids expressing anti-CD19 CARs comprising different hinge domain, transmembrane domain, and/or costimulatory domains. The CAR expression cassettes were all driven by MSCV promoter (see FIG. 1 for vector map). Transfection efficiency was determined using the AF647 conjugated anti-EGFR antibody, and anti-CD19 CAR expression was determined by staining the transduced 293T cells with FITC conjugated CD19 antigen. The data showed that the hinge and transmembrane domain from some transmembrane receptors did not support optimal CAR expression. For example, CD79A hTM-CD28 costim, Long CTLA4 hTM-CD28 costim, and TIM3 hTM-CD28 costim all failed to show optimal CAR expression. In addition, the data suggested that the entire construct with hinge and transmembrane domains and costimulatory domain together were critical for optimal CAR expression. This was based on the observation that combining CD28 costimulatory domain with PD1 or PDL1 hTM resulted in good CAR expression on cell surfaces, but that CD28 costimulatory domains combined with CTLA4-, TIM3-, or CD79A-derived hTM domains each failed to show optimal CAR expression on cell surfaces.

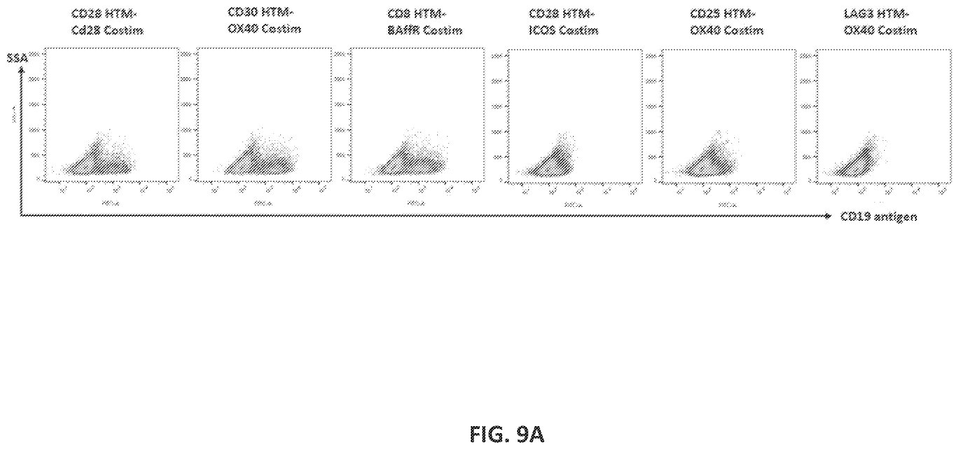

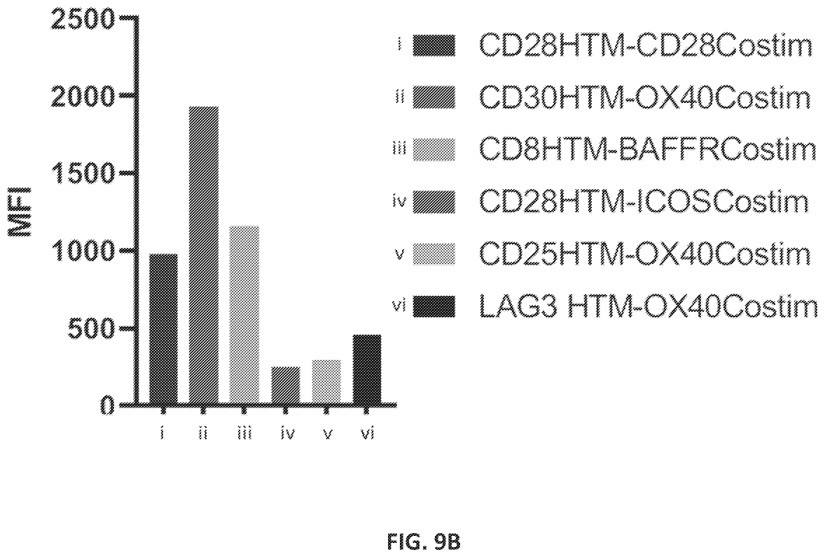

As shown in FIGS. 9A-9B, expression of anti-CD19 CARs with different hinge and transmembrane domains in αβ T cells (e.g., infinite αβ T cells) was determined. The results as depicted in FIG. 9A showed that αβ T cells (e.g., infinite αβ T cells) generated from healthy donor T cells were successfully transduced with lentiviral vectors expressing anti-CD19 CARs of different hinge and transmembrane domain and costimulatory domains. All of the CAR expression cassettes were driven by MSCV promoter. Anti-CD19 CAR expression was determined by staining the transduced T cells with FITC conjugated CD19 antigen. The results showed that CD30 hTM-OX40 costim CAR displayed excellent CAR positive populations, similar to CD28 HTM-CD28 costim and CD8 HTM-BAFF-R costim CARs. Additionally, the results showed that CAR expression was low or absent with other constructs. The anti-CD19 CAR expression on transduced cells displayed in FIG. 9A was quantified as shown in FIG. 9B. The quantification was determined by staining with FITC conjugated CD19 antigen. The MFI of CD30 HTM-OX40 costim CAR population was the highest among all the constructs tested in this experiment. Together these results suggested that the entire construct with hinge and transmembrane domains and costimulatory domain together were important for optimal CAR folding and cell surface expression.

### Example 3—Determining Transduced Cell Cytotoxicity and/or Signaling Levels

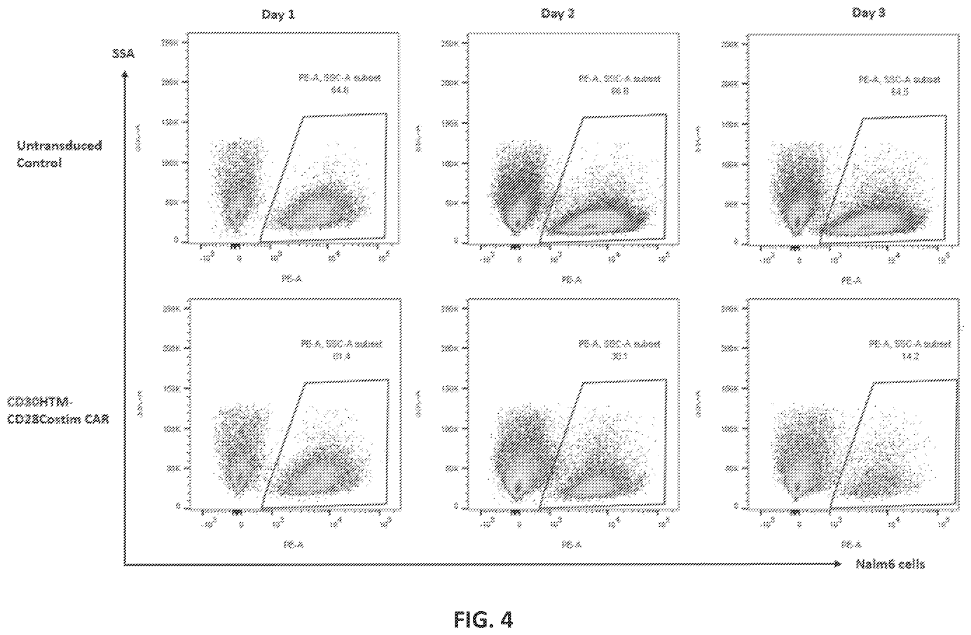

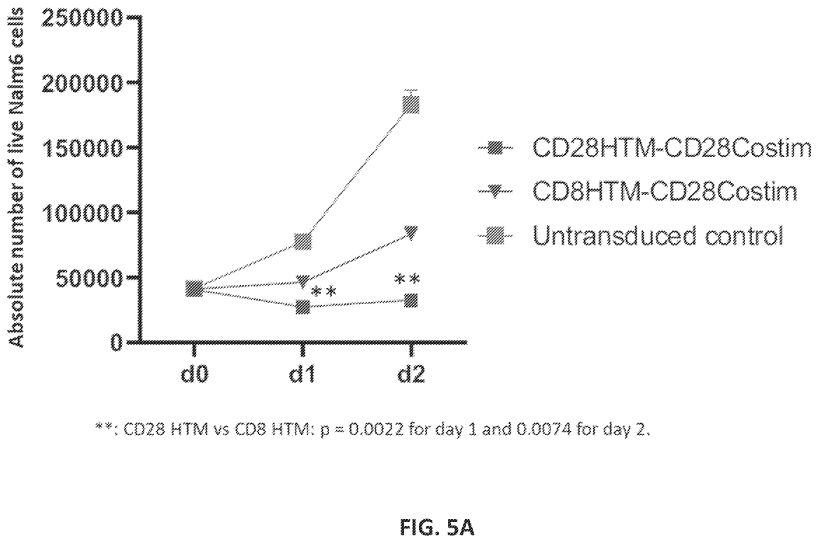

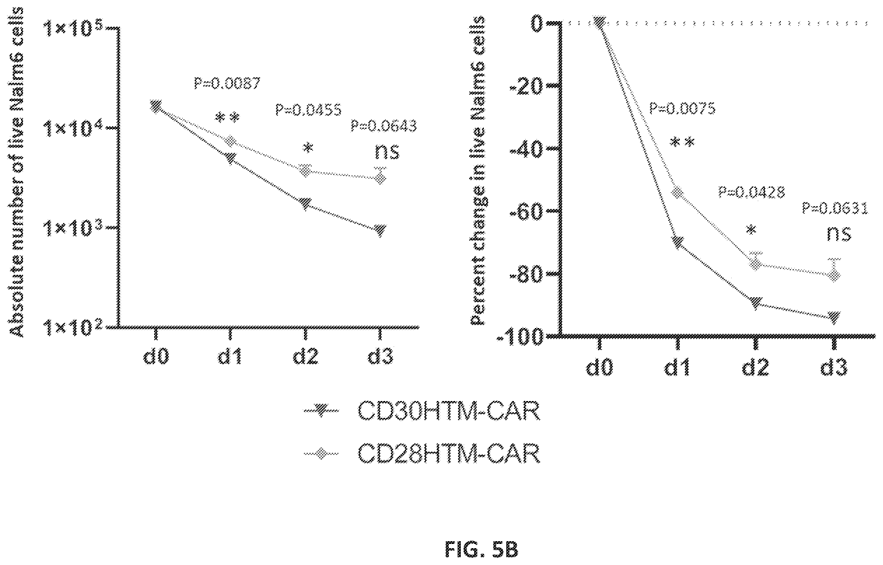

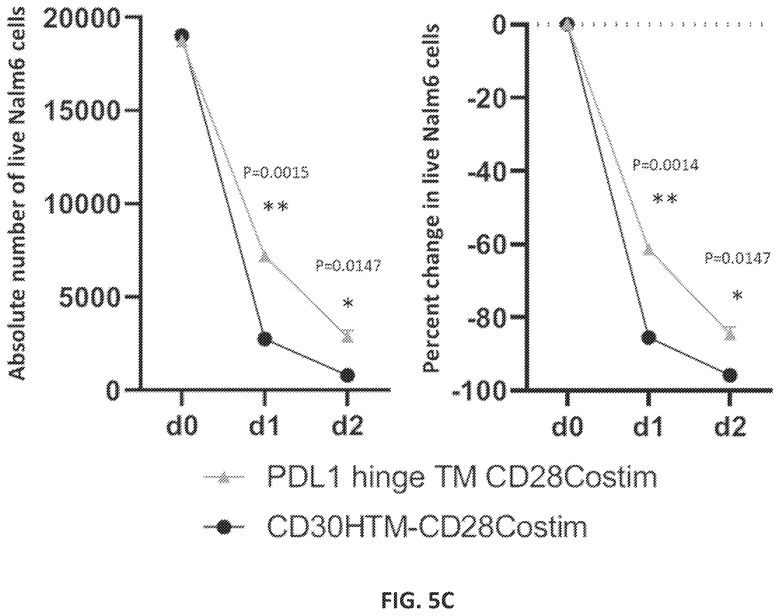

The cytotoxicity of T cells comprising anti-CD19 CAR with CD30hTM-CD28-CD3z against cancer cells was determined. T cells (e.g., infinite γδ T cells) generated from healthy donor T cells were transduced with lentiviral vectors expressing CD30hTM-CD28 costim-based anti-CD19 CAR (tPGK promoter). The CAR expression cassette was driven by a strong composite PGK promoter. After expansion of transduced T cells, the CAR positive percentage was about 20% prior to sorting. These CAR T cells (e.g., CAR infinite γδ T cells) were cocultured with Nalm6 cells with 200 IU/mL of exogenous IL-2 in the medium, and the percentage change in Nalm6 cells was monitored over 3 days. The results showed that the percentage of live Nalm6 cells decreased rapidly over 3 days (see FIG. 4). The cytotoxicity of T cells comprising anti-CD19 CARs with different hinge and transmembrane domains was also determined. T cells (e.g., infinite γδ T cells) generated from healthy donor T cells were transduced with lentiviral vectors expressing anti-CD19 CARs with hinge (h), transmembrane (TM), and costimulatory (costim) domains structured as follows: CD8hTM-CD28 costim, CD28 hTM-CD28 costim, PDL1hTM-CD28 costim, or CD30hTM-CD28 costim respectively (see FIG. 2 for vector maps). After sorting, the CAR positive cells were cocultured with RFP-Luciferase expressing Nalm6 cells. The live cell number of Nalm6 cells was calculated using CountBright™ Absolute Counting Beads on day 0, day 1 and day 2. As shown in FIG. 5A, the CD28 hTM-CD28 costim based anti-CD19 CAR inhibited the proliferation of Nalm6 cells, better than the CD8hTM-CD28 costim based anti-CD19 CAR when cocultured without exogenous IL-2 in the medium. Experiments were also conducted with 200 IU/mL IL-2 in the medium. As shown in FIG. 5B, cells comprising CD30hTM-CD28 costim based anti-CD19 CAR had stronger cytotoxicity than those comprising CD28hTM-CD28 costim based anti-CD19 CAR. As shown in FIG. 5C, cells comprising CD30hTM-CD28 costim based anti-CD19 CAR had stronger cytotoxicity than those comprising PDL1hTM-CD28 costim based anti-CD19 CAR.

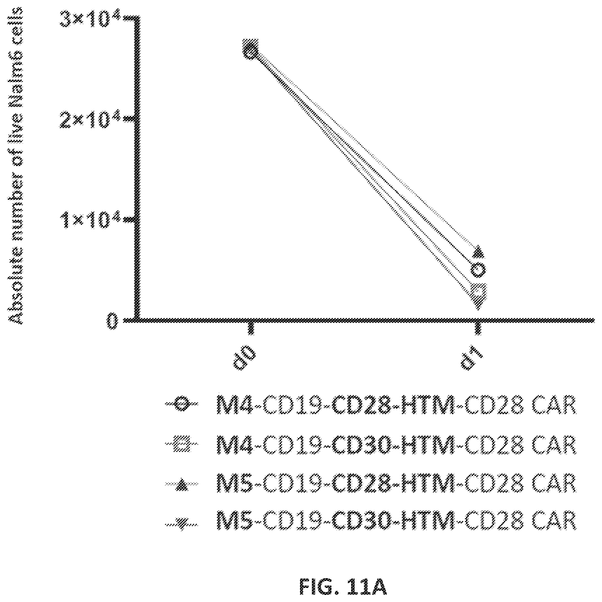

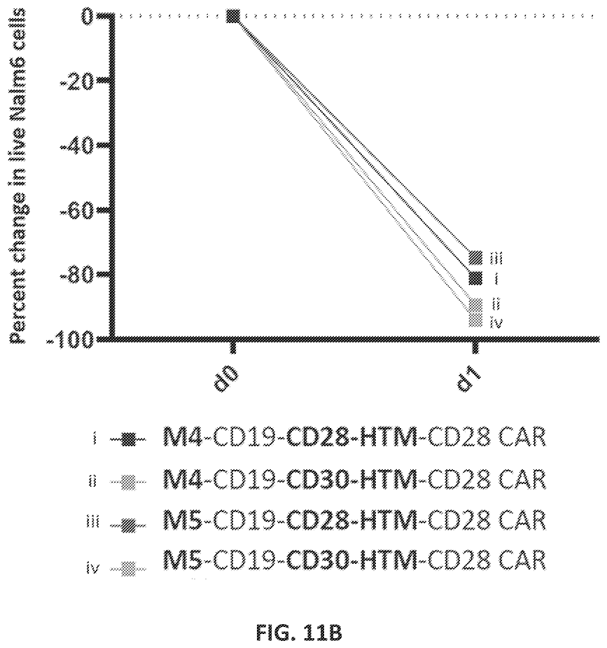

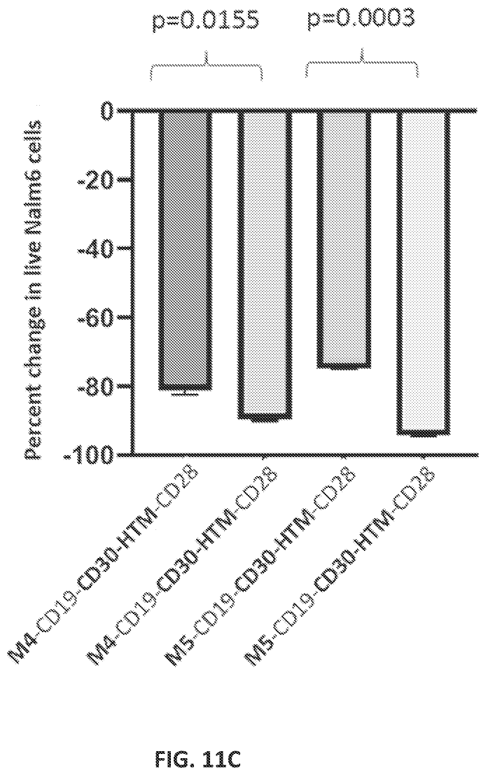

Direct comparisons were drawn between the cytotoxic activity of anti-CD19 CARs with CD28 or CD30 hinge and transmembrane (HTM) domains. T cells (e.g., infinite γδ T cells) generated from healthy donor M4 and donor M5 were transduced with lentiviral vectors expressing anti-CD19 CARs with a CD28 HTM-CD28 costimulatory domain or a CD30HTM-CD28 costimulatory domain respectively, cells were then sorted for CAR+ cells, and were cocultured with luciferase-RFP-expressing Nalm6 acute lymphoblastic leukemia tumor cells in duplicate wells at an Effector:Target ratio of 5:1. The live cell number of Nalm6 cells was calculated using CountBright™ Absolute Counting Beads on day 0 and day 1, shown in FIG. 11A changes in absolute numbers of living Nalm6 cells were obvious. As shown in FIGS. 11B-11C, the percentage of live tumor cells differed as a function of the hinge and transmembrane domain utilized. Data was representative of one of two independent experiments. The results showed that cells comprising a CD19 CAR with CD30 HTM domain had significantly (P values calculated by unpaired T-test) stronger cytotoxicity than cells comprising CD19 CAR with CD28 HTM domain.

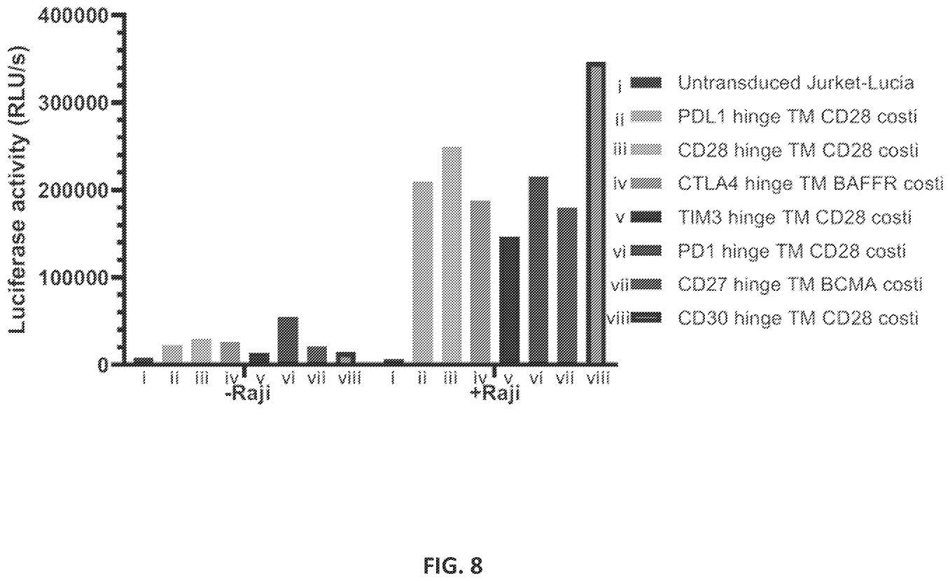

In addition to expression and cytotoxicity, the signaling capability of cells comprising various anti-CD19 CARs with different hinge and transmembrane domains was determined. CAR plasmids which showed acceptable expression in 293T cells (see FIG. 7) were used to produce lentiviral vectors, said lentiviral vectors were used to transfect the Jurkat-Lucia™ NFAT reporter cell line (InvivoGen). The transfected cells were used to quantify CAR-induced signaling by measuring luciferase activity. After sorting the CAR positive populations, each population was cocultured with Raji lymphoma cells at an Effector:Target ratio of 1:1. After 24 hours, luciferase activity was measured in the supernatant (see FIG. 8). The results showed that while all CARs could be specifically activated by Raji cells, cells comprising CD30hTM-CD28 CAR had the highest activity. Additionally, PD1hTM-CD28 CAR had higher tonic signaling than other CARs based on observed activity in the absence of Raji cells.

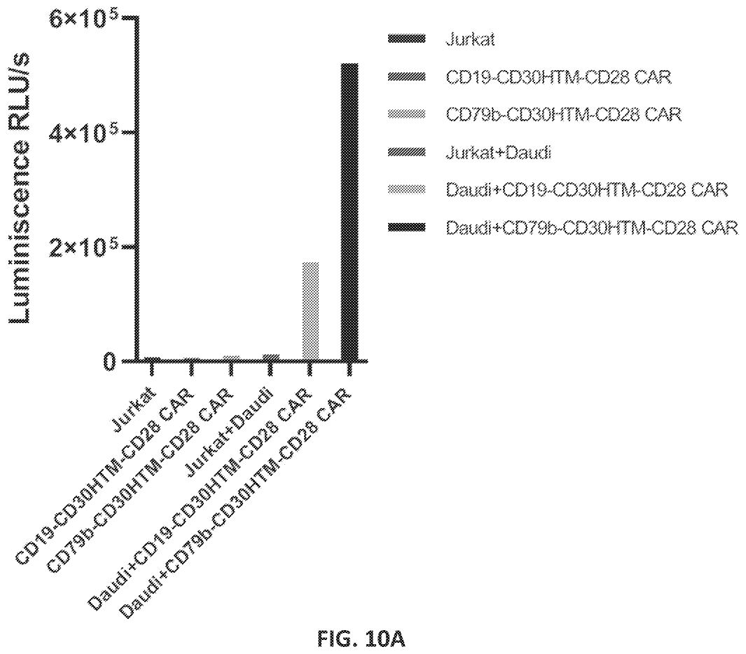

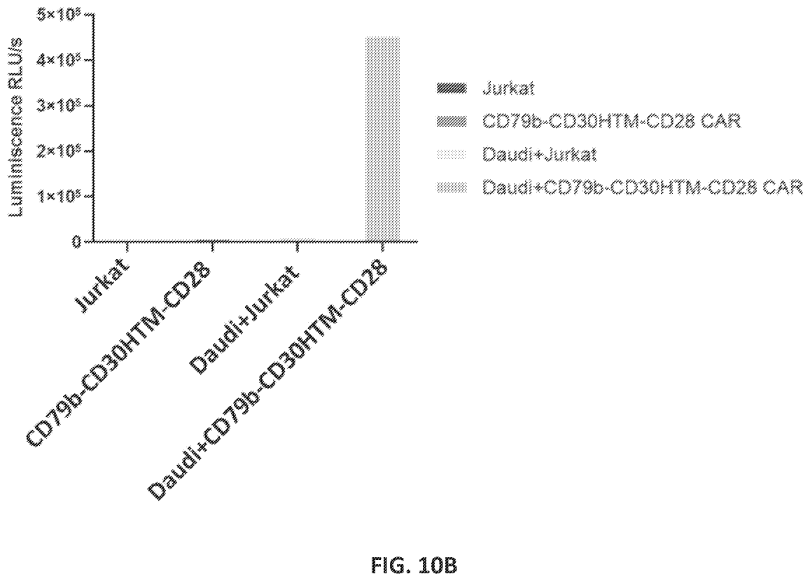

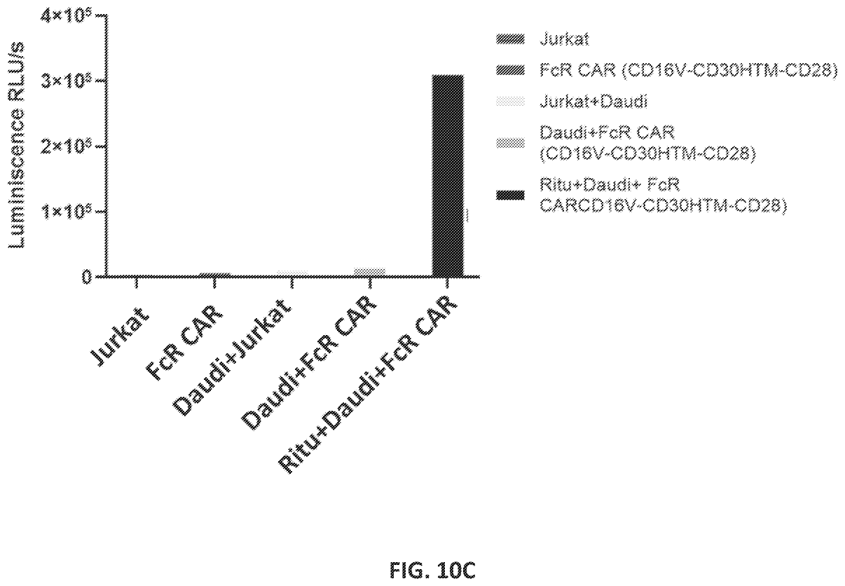

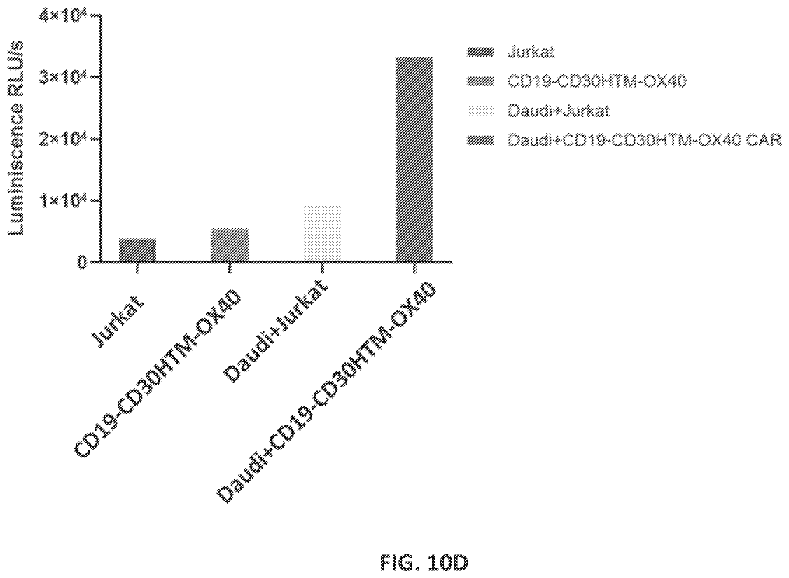

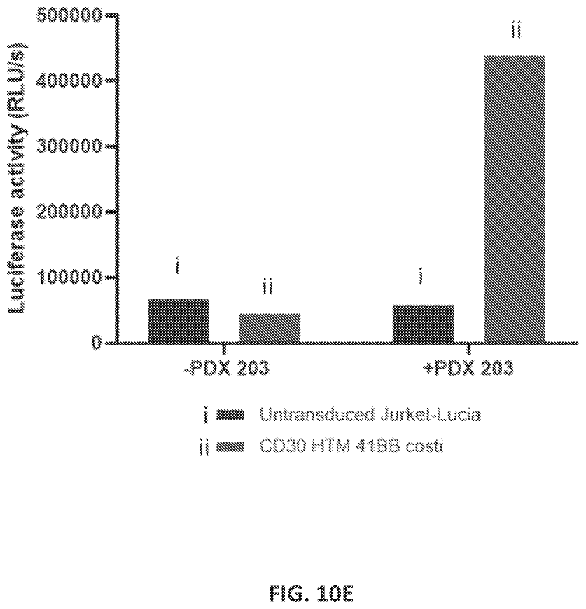

The signaling capabilities of different CARs with CD30 hinge and transmembrane domain (HTM) and CD28-CD3z signaling domains was determined. Lentiviral vectors expressing different CAR constructs were transduced into the Jurkat-Lucia™ NFAT reporter cell line, CAR+ cells were sorted, and CAR-induced signaling was quantified by measuring luciferase activity with or without coculture with Daudi lymphoma cells at an Effector:Target ratio of 1:1. After 24 hours, luciferase activity was measured in the supernatant. As depicted in FIGS. 10A, 10B, and 10D both FMC63 scFv-CD30HTM-CD28costim (CD19-CD30HTM-CD28 CAR) and SN8 scFv-CD30HTM-CD28costim (CD79b-CD30HTM-CD28 CAR) signaled only in the presence of cells that express CD19 and/or CD79b (e.g., Daudi tumor cells). Jurkat-Lucia™ NFAT reporter cells were also transduced with an Fc receptor CAR (FcR CAR-CD16V-CD30HTM-CD28) (see FIG. 10C) the data showed that the FcR CAR only signaled in the presence of both rituximab (an anti-CD20 antibody) and Daudi tumor cells, but not when co-cultured with Daudi tumor cells alone. As shown in FIG. 10E, Jurkat-Lucia™ NFAT reporter cells were also transduced with FMC63 scFv-CD30HTM-CD28costim (CD19-CD30HTM-CD28 CAR) and exposed to high-grade B-cell lymphoma cell lines (PDX 203 lymphoma cells) the data showed that the CD19-CD30HTM-41BB CARs only signaled in the presence of tumor cells. Overall, these results indicate that the CD30 HTM domain functioned as an efficient HTM component with multiple CAR designs targeting different antigens on tumor cells and/or with different costimulatory domains.

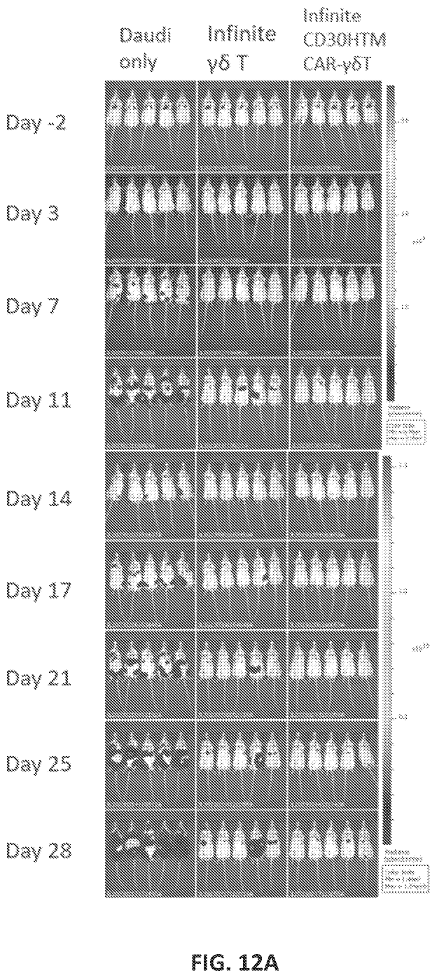

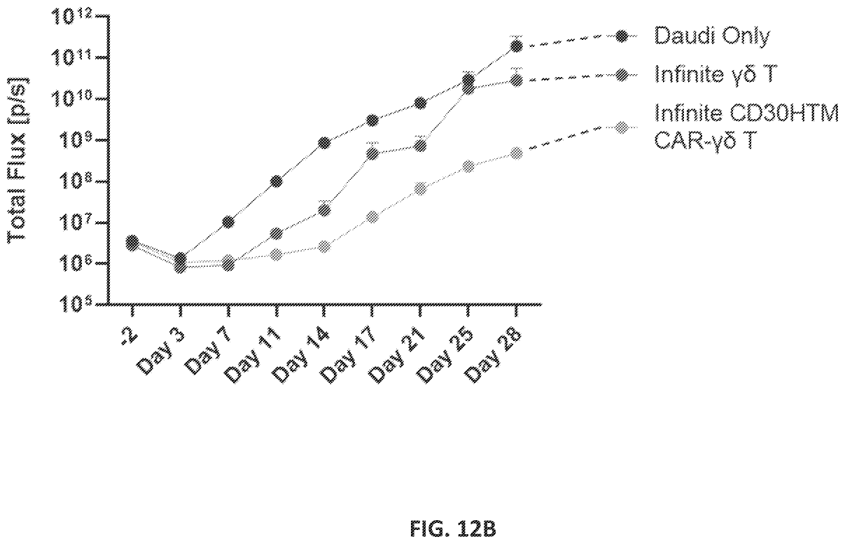

As shown in FIGS. 12A-12B, the in vivo tumor cell control capabilities anti-CD19 CD30HTM CAR-transduced infinite γδ T cells was determined. Luciferase-labeled Daudi Burkitt lymphoma tumor cells (2×10⁴ tumor cells/mouse) were injected intravenously into 3 groups of human IL-15 transgenic NSG mice (secreting physiological level of human IL-15) on day-2. Three infusions of infinite γδ T or infinite anti-CD19 CD30HTM-CD28Cos CAR-γδT were injected into the mice on Days 0, 3, and 8 at a dose of 8×10⁶ T cells/mouse/injection. The data showed that the CD30HTM-CD28Cos CAR-γδT could slow down the progression of lymphoma. Tumor burden was assessed by bioluminescence imaging (FIG. 12A) and mean total flux in each group was calculated and shown (FIG. 12B). The data indicate that the antitumor effect of infinite γδ CAR T cells comprising a CD30HTM was higher than infinite γδ T cells without a CAR.

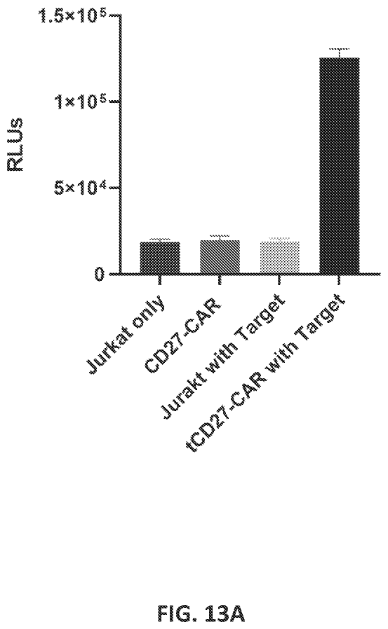

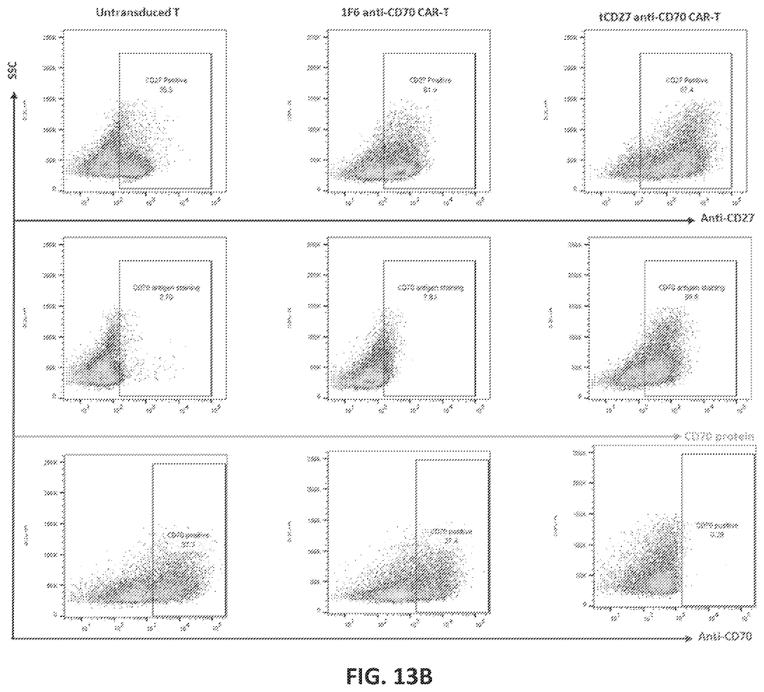

As shown in FIGS. 13A-13B, CD70 binding CARs comprising truncated CD27 (tCD27) CAR with CD30 hinge and transmembrane domains were successfully transduced, expressed, induced to signal, and bind to CD70. FIG. 13A, shows how a CD27-based anti-CD70 CAR was made by fusion of the truncated CD27 extracellular domain (SEQ ID NO: 52 encoding SEQ ID NO: 48) with the CD30 hinge and TM domains, CD28 costimulatory domain, and CD3z signaling domain. The signaling capability was determined using Jurkat-Lucia™ NFAT reporter cell line (Invivogen). Lentiviral vectors expressing this CAR construct (SEQ ID NO: 49) was transduced into the Jurkat-Lucia™ NFAT reporter cell line, CAR+ cells were sorted, and CAR-induced signaling was quantified by measuring luciferase activity with or without coculture with a CD70 positive T cell line at an Effector:Target ratio of 1:1. After 24 hours, luciferase activity was measured in the supernatant. As shown, the tCD27-CD30HTM-CD28cos-CD3z CAR signaled only in the presence of cells that expressed CD70. The results indicated that the CD30 HTM domain functioned as an efficient HTM component with multiple CAR designs targeting different antigens on tumor cells and/or with different costimulatory domains. RLU=relative light units. FIG. 13B, shows expression of the aforementioned lentiviral vectors (tCD27-CD30HTM-CD28cos-CD3z CAR) transduced into primary human T cells. Ten days after transduction, CD27 and CD70 cell surface expression on CAR-T cells was measured. The CAR's ability to bind to CD70 was also tested for by staining with a fluorochrome conjugated recombinant CD70 protein. Non-transduced T cells and a low affinity anti-CD70 scFv antibody (Clone 1F6) were utilized as controls. The anti-CD27 antibody staining results indicated that tCD27 folded appropriately and was expressed on the surface of primary T cells (top row). The CD70 protein staining indicated that the tCD27 CAR bound recombinant CD70 protein (middle row). The anti-CD70 antibody staining showed that the CD70+ cells were nearly absent in tCD27 CAR-transduced T cells, indicating that cells expressing CD70 were either efficiently eliminated, or that CD70 on cell surfaces was masked through in cis binding (bottom row).

Together, these results indicated that CD30 HTM domain functioned as an efficient HTM component with multiple CAR designs targeting different antigens on tumor cells in vitro and in vivo.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

References, cited herein, to the extent that they provide exemplary procedural or other details supplementary to those set forth herein, are specifically incorporated herein by reference.
U.S. Pat. No. 4,554,101
U.S. Pat. No. 4,684,611
U.S. Pat. No. 4,952,500
U.S. Pat. No. 5,302,523
U.S. Pat. No. 5,322,783
U.S. Pat. No. 5,384,253
U.S. Pat. No. 5,464,765
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,589,466
U.S. Pat. No. 5,591,616
U.S. Pat. No. 5,610,042
U.S. Pat. No. 5,656,610
U.S. Pat. No. 5,677,425
U.S. Pat. No. 5,702,932
U.S. Pat. No. 5,736,524
U.S. Pat. No. 5,780,448
U.S. Pat. No. 5,789,215
U.S. Pat. No. 5,945,100
U.S. Pat. No. 5,981,274
U.S. Pat. No. 5,994,624
U.S. Pat. No. 6,410,319
U.S. Pat. No. 6,544,518
WO/2021/034982
WO2021/034982
WO2021/222944
WO94/09699
WO95/06128
WO99/40188
Ausubel et al., Current Protocols in Molecular Biology, Greene Publishing Associates and John Wiley & Sons, NY (1994)
Burton, Molec. Immunol., 22:161-206 (1985)
Chen and Okayama (1987)
Fechheimer et al. (1987)
Fraley et al. (1979)
Frolet et al. (2010)
Graham and Van Der Eb (1973)
Harland and Weintraub (1985)
Huck et al., Nucl. Acids Res (1986)
Kaeppler et al. (1990)
Kaneda et al. (1989)
Kato et al. (1991)
Kyte et al., J. Mol. Biol. 157:105-131 (1982)
Nicolau and Sene (1982)
Nicolau et al. (1987)
Omirulleh et al. (1993)
Potrykus et al. (1985)
Rieder et al. (1999)
Rippe et al. (1990)
Romain Studer et al., Biochem. J. 449:581-594 (2013)
Sambrook et al., Molecular Cloning: A Laboratory Manual, 3rd ed., Cold Spring Harbor Press, (2001)
Snyder et al., PATRIC: The VBI PathoSystems (2007)
Tan et al., Proc. Natl. Acad. Sci. USA87: 162 (1990)
Wong et al. (1980)
Zysk et al. (2000)

What is claimed is:

1. A Chimeric Antigen Receptor (CAR) comprising:
i) an antigen binding domain;
ii) a CD30 hinge domain;
iii) a CD30 transmembrane domain;
iv) at least one intracellular costimulatory domain; and
v) an intracellular stimulatory domain;
wherein the CD30 hinge domain comprises less than 51 contiguous amino acids and at least 7 contiguous amino acids of the extracellular domain of CD30; and
wherein the CD30 transmembrane domain comprises no more than 27 contiguous amino acids of CD30.

2. The CAR of claim 1, wherein the CD30 hinge and/or transmembrane domain do not comprise a cysteine.

3. The CAR of claim 1 or 2, wherein the CD30 hinge is at least 80%, 85%, 90%, 95%, or 98% identical to SEQ ID NO: 3.

4. The CAR of any one of claims 1-3, wherein the CD30 transmembrane domain is at least 80%, 85%, 90%, or 95% identical to SEQ ID NO: 4.

5. The CAR of any one of claims 1-4, wherein the CD30 hinge domain and/or transmembrane domain lack 3 or more contiguous amino acids according to SEQ ID NO: 6.

6. The CAR of any one of claims 1-5, wherein the CD30 hinge domain comprises SEQ ID NO: 3.

7. The CAR of any one of claims 1-6, wherein the CD30 transmembrane domain comprises SEQ ID NO: 4.

8. The CAR of any one of claims 1-7, wherein the CD30 domain and transmembrane domain are encoded by a nucleotide sequence at least 75%, 80%, 85%, 90%, 95%, or 98% identical to SEQ ID NO: 39.

9. The CAR of any one of claims 1-8, wherein the antigen binding domain is targeted to CD19, CD20, CD22, CD70, CD79B, CD79A, ROR1, BCMA, BAFF receptor, GD2, or claudin18.2.

10. The CAR of claim 9, wherein the antigen binding domain is targeted to CD19, CD79B, and/or CD70.

11. The CAR of any one of claims 1-9, wherein the at least one intracellular costimulatory domain comprises a CD8, CD27, CD28, CD30, CD38, CD35, CD45, CD4, CD5, CD9, CD16, CD22, CD33, CD37, CD64, CD80, CD86, CD134 (OX40), CD137 (4-1BB), or CD154, intracellular costimulatory domain.

12. The CAR of claim 11, wherein the at least one intracellular costimulatory domain comprises CD28.

13. The CAR of any one of claims 1-12, wherein the intracellular stimulatory domain comprises a DAP12, DAP10, FCER1G (Fc epsilon receptor I gamma chain), CD3δ (CD3 delta), CD3ε (CD3 epsilon), CD3γ (CD3 gamma), CD3ζ (CD3 zeta), or CD79A, intracellular stimulatory domain.

14. The CAR of claim 13, wherein the intracellular stimulatory domain comprises CD35 (CD3 zeta).

15. The CAR of any one of claims 1-14, wherein the CD30 hinge domain, CD30 transmembrane domain, at least one intracellular costimulatory domain, and intracellular stimulatory domain are at least 85%, 90%, 95%, 99%, or 100% identical to SEQ ID NO: 2.

16. A method of producing a cell comprising transducing and/or transfecting a cell with the CAR of any one of claims 1-15.

17. A cell produced according to claim 16.

18. The cell of claim 17, wherein the cell is a T cell or an NK cell.

19. The cell of claim 18, wherein the cell is a γδ T cell.

20. The cell of any one of claims 17-19, wherein the cell comprises the BCL6 gene and at least one additional transgene encoding an immunomodulatory gene.

21. The cell of claim 20, wherein the at least one additional transgene encoding an immunomodulatory gene is a Bcl2 family gene.

22. The cell of any one of claims 17-21, wherein the cell comprises at least one manmade mutation in an endogenous gene, and/or at least one heterologous nucleic acid that can modify expression of at least one endogenous gene.

23. The cell of claim 22, wherein the endogenous gene is an immunomodulatory gene.

24. A composition comprising the cell of any one of claims 17-23.

25. A method of treating a hematological cancer in a patient comprising administration to the patient the cell of any one of claims 17-23 or the composition of claim 24.

26. The method of claim 25, further comprising administering at least a second therapeutic agent to the subject.

27. The method of claim 26, wherein the at least a second therapeutic agent comprises chemotherapy, immunotherapy, surgery, radiotherapy, drug therapy, targeted therapy, hormone therapy, biotherapy, or a combination thereof.


SEQUENCE LISTING (52 sequences)

SEQ ID NO: 1 - protein, 63 aa, Homo sapiens
APASTSPTQSLLVDSQASKTLPIPTSAPVALSSTGKPVLDAGPVLFWVILVLVVVVGSSAFLL

SEQ ID NO: 2 - protein, 220 aa, synthetic construct
APASTSPTQSLLVDSQASKTLPIPTSAPVALSSTGKPVLDAGPVLFWVILVLVVVVGSSAFLLYCWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 42 aa, Homo sapiens
APASTSPTQSLLVDSQASKTLPIPTSAPVALSSTGKPVLDAG

SEQ ID NO: 4 - protein, 21 aa, Homo sapiens
PVLFWVILVLVVVVGSSAFLL

SEQ ID NO: 5 - protein, 18 aa, Homo sapiens
APPLGTQPDCNPTPENGE

SEQ ID NO: 6 - protein, 367 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 7 - protein, 66 aa, Homo sapiens
IEVMYPPPYLDNEKSNGTIIHVKGKHLCPSPLFPGPSKPFWVLVVVGGVLACYSLLVTVAFIIFWV

SEQ ID NO: 8 - protein, 66 aa, Homo sapiens
TTTPAPRPPTPAPTIASQPLSLRPEACRPAAGGAVHTRGLDFACDIYIWAPLAGTCGVLLLSLVIT

SEQ ID NO: 9 - protein, 421 aa, synthetic construct
MLLLVTSLLLCELPHPAFLLIGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITVSSAPASTSPTQSLLVDSQASKTLPIPTSAPVALSSTGKPVLDAGPVLFWVILVLVVVVGSSAFLLYCWVRSKRSRLLHSDYMNMTPRRPGPTRKHYQPYAPPRDFAAYRSRVKFSRSADAITSYEKSDGVYTGLSTRNQETYETLKHEKPPQAIRKRQPEETNNDYETADGGYMTLNPRAPTDDDKNIYLTLPPNDHVNSNNYTGLSTRNQETYETLKHEKPPQ

SEQ ID NO: 10 - protein, 32 aa, Homo sapiens
ITSYEKSDGVYTGLSTRNQETYETLKHEKPPQ

SEQ ID NO: 11 - protein, 22 aa, Homo sapiens
YTGLSTRNQETYETLKHEKPPQ

SEQ ID NO: 12 - protein, 70 aa, synthetic construct
VSSAPASTSPTQSLLVDSQASKTLPIPTSAPVALSSTGKPVLDAGPVLFWVILVLVVVVGSSAFLLYCWV

SEQ ID NO: 13 - protein, 175 aa, Homo sapiens
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITIT

SEQ ID NO: 14 - protein, 21 aa, Homo sapiens
MLLLVTSLLLCELPHPAFLLI

SEQ ID NO: 15 - protein, 41 aa, Homo sapiens
RSKRSRLLHSDYMNMTPRRPGPTRKHYQPYAPPRDFAAYRS

SEQ ID NO: 16 - protein, 10 aa, Homo sapiens
RVKFSRSADA

SEQ ID NO: 17 - protein, 50 aa, Homo sapiens
AIRKRQPEETNNDYETADGGYMTLNPRAPTDDDKNIYLTLPPNDHVNSNN

SEQ ID NO: 18 - protein, 483 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAPASTSPTQSLLVDSQASKTLPIPTSAPVALSSTGKPVLDAGPVLFWVILVLVVVVGSSAFLLYCWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 21 aa, Homo sapiens
MALPVTALLLPLALLLHAARP

SEQ ID NO: 20 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 21 - protein, 67 aa, synthetic construct
APASTSPTQSLLVDSQASKTLPIPTSAPVALSSTGKPVLDAGPVLFWVILVLVVVVGSSAFLLYCWV

SEQ ID NO: 22 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 491 aa, synthetic construct
MALPVTALLLPLALLLHAARPDIVLTQSPASLAVSLGQRATISCKASQSVDYDGDSFLNWYQQKPGQPPKLFIYAASNLESGIPARFSGSGSGTDFTLNIHPVEEEDAATYYCQQSNEDPLTFGAGTELELKRSGGGGSGGGGSGGGGSGGGGSEVQLQQSGAELMKPGASVKISCKATGYTFSSYWIEWVKQRPGHGLEWIGEILPGGGDTNYNEIFKGKATFTADTSSNTAYMQLSSLTSEDSAVYYCTRRVPVYFDYWGQGTSVTVSSAPASTSPTQSLLVDSQASKTLPIPTSAPVALSSTGKPVLDAGPVLFWVILVLVVVVGSSAFLLYCWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 250 aa, synthetic construct
DIVLTQSPASLAVSLGQRATISCKASQSVDYDGDSFLNWYQQKPGQPPKLFIYAASNLESGIPARFSGSGSGTDFTLNIHPVEEEDAATYYCQQSNEDPLTFGAGTELELKRSGGGGSGGGGSGGGGSGGGGSEVQLQQSGAELMKPGASVKISCKATGYTFSSYWIEWVKQRPGHGLEWIGEILPGGGDTNYNEIFKGKATFTADTSSNTAYMQLSSLTSEDSAVYYCTRRVPVYFDYWGQGTSVTVSS

SEQ ID NO: 25 - protein, 42 aa, synthetic construct
KRGRKKLLYIFKQPFMRPVQTTQEEDGCSCRFPEEEEGGCEL

SEQ ID NO: 26 - protein, 37 aa, Homo sapiens
RRDQRLPPDAHKPPGGGSFRTPIQEEQADAHSTLAKI

SEQ ID NO: 27-29 - skipped (3 entries)

SEQ ID NO: 30 - protein, 10 aa, synthetic construct
EQKLISEEDL

SEQ ID NO: 31-38 - skipped (8 entries)

SEQ ID NO: 39 - DNA, 189 bp, Homo sapiens
  source 1..189: mol_type = other DNA
gcccccgcttctacttcaccaacccagagccttctagtagacagtcaggcctccaagacgctgcccatccctacctccgccccggtggctctgagctcgaccggcaagcccgtgctggatgcaggtcccgtgctcttttgggtcattctggtgctggtggtggtcgttggctcctctgcgttcctgctg

SEQ ID NO: 40 - DNA, 1263 bp, synthetic construct
  source 1..1263: mol_type = other DNA
atgctgctgctggtgacatccctgctgctgtgcgagctgccccatcccgcctttctgctgattggaatgaggactgaggacctgcccaaggccgtggtgttcctggagccccagtggtaccgggtgctggagaaggacagcgtgaccctgaagtgccagggcgcctattcccctgaggataactctacacagtggttccacaatgagtctctgatcagctcccaggcctctagctactttatcgacgcagcaaccgtggacgatagcggagagtatcggtgccagaccaacctgagcacactgtccgatcctgtgcagctggaggtgcacatcggatggctgctgctgcaggccccaagatgggtgtttaaggaggaggaccctatccacctgagatgtcactcctggaagaataccgccctgcacaaggtgacatacctgcagaacggcaagggcaggaagtacttccaccacaatagcgacttttatatcccaaaggccaccctgaaggattctggcagctacttctgcaggggcctggtgggctccaagaacgtgtcctctgagacagtgaatatcaccatcacagtcagcagtgcaccggcttccacgtcccccacccaaagcttgctggtagactcccaagccagtaagacacttcccataccaacaagcgcgccggtagcgttgtcatccacgggaaaacccgtcttggacgcagggccagtcctgttttgggtaattctggttttggtcgttgtggttggctcaagtgcgttcctcctgtattgctgggttcgaagtaaacgctcaaggcttcttcactccgactatatgaatatgaccccgaggagacctgggccaacgcggaaacattatcaaccctatgccccccctagggactttgcggcttataggtcccgcgtaaagtttagccggagcgcagacgccatcacctcttacgagaagagcgacggcgtttacacggggctgtctaccaggaatcaagagacttacgaaacccttaaacatgaaaaacccccacaggccatccgaaaacgacagcccgaagaaacaaataatgactacgagaccgccgatggaggatatatgactcttaaccctagggctcctaccgatgacgataaaaatatctacttgacactgcctcccaacgaccatgtcaacagtaacaattatactgggctgtcaacccggaatcaagaaacgtatgagacgttgaaacatgagaagcccccacaa

SEQ ID NO: 41 - DNA, 1449 bp, synthetic construct
  source 1..1449: mol_type = other DNA
atggccctgccagtgaccgccctgctgctgccactggcactgctgctgcacgcagcaaggccagacatccagatgacacagaccacaagctccctgtccgcctctctgggcgacagagtgaccatctcttgcagggccagccaggatatctccaagtatctgaattggtaccagcagaagcctgatggcacagtgaagctgctgatctatcacacctctagactgcacagcggcgtgccatccaggtttagcggctccggctctggcacagactactctctgaccatcagcaatctggagcaggaggatatcgccacctatttctgccagcagggcaacacactgccttacacctttggcggcggcacaaagctggagatcaccggcggcggcggctctggaggaggaggaagcggaggaggaggatccgaggtgaagctgcaggagagcggaccaggactggtggcacccagccagtccctgtctgtgacatgtaccgtgtccggcgtgtctctgccagactacggcgtgagctggatcagacagccacctaggaagggtctggagtggctaggtgtgatttggggctccgagaccacatattacaacagcgcgctcaagtcccgcctgaccattatcaaggacaactccaagtcgcaggtgttcctgaagatgaattccttgcagactgatgacaccgccatctattactgcgccaagcattactactacggcggtagttacgccatggattattggggccagggcacctccgtgaccgtaagctcggcccccgcttctacttcaccaacccagagccttctagtagacagtcaggcctccaagacgctgcccatccctacctccgccccggtggctctgagctcgaccggcaagcccgtgctggatgcaggtcccgtgctcttttgggtcattctggtgctggtggtggtcgttggctcctctgcgttcctgctgtattgttgggtgagaagcaagagatccaggctgctgcacagcgactacatgaatatgacaccaaggagaccaggaccaaccaggaagcactatcagccttacgcacctccaagggacttcgcagcatataggagccgtgtgaaatttagccggtccgcggacgcccctgcttatcagcagggacagaaccagctgtacaacgagctcaacctgggtcgcagggaggagtacgacgtgctggacaagcgccgcggccgcgacccagaaatgggcgggaagcccaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctaga

SEQ ID NO: 42 - DNA, 1473 bp, synthetic construct
  source 1..1473: mol_type = other DNA
atggccctgccagtgaccgccctgctgctgccactggcactgctgctgcacgcagcaaggccagacattgtgctgacacagtcccctgcaagcctcgctgtatctctcggtcagagggccacaatatcatgtaaggcgagtcaatccgttgactatgatggagatagctttttgaactggtaccaacagaagcccggacagccaccgaagctcttcatttacgcagcatcaaatcttgagtctggaataccagcacggtttagcggatctgggagcggcacagattttacgttgaatatacatcccgtggaagaggaagacgcagcaacgtactattgtcaacagtctaatgaggatccattgacgttcggagcaggcacagagctcgaacttaagaggagcggaggaggaggctccggtggaggtggatctggtggtggaggcagcggaggcggaggctcagaggtacaacttcagcagtccggcgctgaactgatgaagccaggggcgtccgttaaaatttcttgtaaagcgacagggtatacctttagttcttactggattgaatgggtcaaacaaagaccaggacatggactcgaatggattggggagatcttgccaggaggaggggacaccaactacaacgagatcttcaaggggaaggcgacgttcactgcagatactagcagcaacaccgcttatatgcagctcagttcacttacgtcagaggacagcgccgtgtattattgcaccaggagagttcctgtttattttgactattggggtcagggaacttccgttactgtgtccagtgcccccgcttctacttcaccaacccagagccttctagtagacagtcaggcctccaagacgctgcccatccctacctccgccccggtggctctgagctcgaccggcaagcccgtgctggatgcaggtcccgtgctcttttgggtcattctggtgctggtggtggtcgttggctcctctgcgttcctgctgtattgttgggtgagaagcaagagatccaggctgctgcacagcgactacatgaatatgacaccaaggagaccaggaccaaccaggaagcactatcagccttacgcacctccaagggacttcgcagcatataggagccgtgtgaaatttagccggtccgcggacgcccctgcttatcagcagggacagaaccagctgtacaacgagctcaacctgggtcgcagggaggagtacgacgtgctggacaagcgccgcggccgcgacccagaaatgggcgggaagcccaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctaga

SEQ ID NO: 43 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGPEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 44 - DNA, 2118 bp, Homo sapiens
  source 1..2118: mol_type = other DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 45 - protein, 706 aa, Homo sapiens
MASPADSCIQFTRHASDVLLNLNRLRSRDILTDVVIVVSREQFRAHKTVLMACSGLFYSIFTDQLKCNLSVINLDPEINPEGFCILLDFMYTSRLNLREGNIMAVMATAMYLQMEHVVDTCRKFIKASEAEMVSAIKPPREEFLNSRMLMPQDIMAYRGREVVENNLPLRSAPGCESRAFAPSLYSGLSTPPASYSMYSHLPVSSLLFSDEEFRDVRMPVANPFPKERALPCDSARPVPGEYSRPTLEVSPNVCHSNIYSPKETIPEEARSDMHYSVAEGLKPAAPSARNAPYFPCDKASKEEERPSSEDEIALHFEPPNAPLNRKGLVSPQSPQKSDCQPNSPTESCSSKNACILQASGSPPAKSPTDPKACNWKKYKFIVLNSLNQNAKPEGLEQAELGRLSPRAYTAPPACQPPMEPENLDLQSPTKLSASGEDSTIPQASRLNNIVNRSMTGSPRSSSESHSPLYMHPPKCTSCGSQSPQHAEMCLHTAGPTFPEEMGETQSEYSDSSCENGAFFCNECDCRFSEEASLKRHTLQTHSDKPYKCDRCQASFRYKGNLASHKTVHTGEKPYRCNICGAQFNRPANLKTHTRIHSGEKPYKCETCGARFVQVAHLRAHVLIHTGEKPYPCEICGTRFRHLQTLKSHLRIHTGEKPYHCEKCNLHFRHKSQLRLHLRQKHGAITNTKVQYRVSATDLPPELPKAC

SEQ ID NO: 46 - DNA, 2118 bp, Homo sapiens
  source 1..2118: mol_type = other DNA
atggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcttgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgc

SEQ ID NO: 47 - protein, 191 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 48 - protein, 124 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLP

SEQ ID NO: 49 - protein, 344 aa, synthetic construct
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPAPASTSPTQSLLVDSQASKTLPIPTSAPVALSSTGKPVLDAGPVLFWVILVLVVVVGSSAFLLYCWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 19 aa, Homo sapiens
MARPHPWWLCVLGTLVGLS

SEQ ID NO: 51 - DNA, 1032 bp, synthetic construct
  source 1..1032: mol_type = other DNA
atggcaaggccacacccttggtggctgtgtgtactggggactcttgtaggactgtccgcgacccctgccccgaagagctgtcccgaacgacattattgggctcaaggaaagttgtgctgtcaaatgtgcgaaccgggtacgtttctcgttaaggattgtgaccagcatcggaaagctgcccaatgcgacccttgtatccccggagtttctttttcaccagatcaccatacgaggccgcactgcgagtcatgccggcactgcaatagcggccttcttgtgaggaactgcacaataaccgcaaatgcagagtgtgcctgtagaaatggttggcaatgccgggataaagaatgtacggaatgtgaccctctgccagcccccgcttctacttcaccaacccagagccttctagtagacagtcaggcctccaagacgctgcccatccctacctccgccccggtggctctgagctcgaccggcaagcccgtgctggatgcaggtcccgtgctcttttgggtcattctggtgctggtggtggtcgttggctcctctgcgttcctgctgtattgttgggtgagaagcaagagatccaggctgctgcacagcgactacatgaatatgacaccaaggagaccaggaccaaccaggaagcactatcagccttacgcacctccaagggacttcgcagcatataggagccgtgtgaaatttagccggtccgcggacgcccctgcttatcagcagggacagaaccagctgtacaacgagctcaacctgggtcgcagggaggagtacgacgtgctggacaagcgccgcggccgcgacccagaaatgggcgggaagcccaggcgcaagaacccacaggagggcctgtataatgagctgcagaaggacaagatggccgaggcctacagcgagatcggcatgaagggagagaggagaaggggcaagggacacgatggcctgtatcagggcctgtccacagccaccaaggacacctacgatgcactgcacatgcaggcactgccacctaga

SEQ ID NO: 52 - DNA, 372 bp, synthetic construct
  source 1..372: mol_type = other DNA
atggcaaggccacacccttggtggctgtgtgtactggggactcttgtaggactgtccgcgacccctgccccgaagagctgtcccgaacgacattattgggctcaaggaaagttgtgctgtcaaatgtgcgaaccgggtacgtttctcgttaaggattgtgaccagcatcggaaagctgcccaatgcgacccttgtatccccggagtttctttttcaccagatcaccatacgaggccgcactgcgagtcatgccggcactgcaatagcggccttcttgtgaggaactgcacaataaccgcaaatgcagagtgtgcctgtagaaatggttggcaatgccgggataaagaatgtacggaatgtgaccctctgcca